SEQUENCE LISTING (29 sequences)

SEQ ID NO: 1 - protein, 11 aa, unidentified
YPDEVKRKKKP

SEQ ID NO: 2 - protein, 38 aa, synthetic construct
SLLESPFDKPDEVKRKKKPPTSHQSDATAEDDSSSKKK

SEQ ID NO: 3 - DNA, 23 bp, synthetic construct
  source 1..23: mol_type = other DNA
tagaggtaacaactagcgtacaa

SEQ ID NO: 4 - DNA, 33 bp, synthetic construct
  source 1..33: mol_type = other DNA
tagaggtaacaactagcgtacaatttttttttt

SEQ ID NO: 5 - protein, 4 aa, synthetic construct
GGGG

SEQ ID NO: 6 - protein, 12 aa, synthetic construct
QPRRRPRRKKRG

SEQ ID NO: 7 - protein, 5 aa, synthetic construct
  REPEAT 1..5: note = The sequence may be present 1, 2, 3, 4, or 5 times
GGGGS

SEQ ID NO: 8 - DNA, 33 bp, synthetic construct; identical to SEQ ID NO: 4
  source 1..33: mol_type = other DNA
  misc_feature 33: note = The last tyhmine of SEQ ID NO: 8 is appended to a polypeptide sequence of SEQ ID NO: 28 (HHHHHHC)

SEQ ID NO: 9 - protein, 17 aa, synthetic construct
  DNA_BIND 1: note = The N-terminal glycine of SEQ ID NO: 9 is appended to the 3' terminal thymine of SEQ ID NO: 4 (5'-TAGAGGTAACAACTAGCGTACAATTTTTTTTTT-3')
GGGGGGYPDEVKRKKKP

SEQ ID NO: 10 - protein, 34 aa, synthetic construct
  DNA_BIND 1: note = The N-terminal glycine of SEQ ID NO: 10 is appendend to the 3' terminal thymine of SEQ ID NO: 4 (5'-TAGAGGTAACAACTAGCGTACAATTTTTTTTTT-3')
  SITE 7: note = X is citroline ("Cit")
GGGGGGXVGGGGYPDEVKRKKKPGGGGHHHHHHC

SEQ ID NO: 11 - protein, 69 aa, synthetic construct
  DNA_BIND 1: note = The N-terminal glycine of SEQ ID NO: 11 is appended to the 3' terminal thymine of SEQ ID NO: 4 (5'-TAGAGGTAACAACTAGCGTACAATTTTTTTTTT-3')
  SITE 7: note = X is citroline ("Cit")
GGGGGGXVQPRRRPRRKKRGSLLESPFDKPDEVKRKKKPPTSHQSDATAEDDSSSKKKGGGGHHHHHHC

SEQ ID NO: 12 - protein, 32 aa, synthetic construct
ATPKKSTKKTPKKAKKATPKKSTKKTPKKAKK

SEQ ID NO: 13 - protein, 10 aa, synthetic construct
WRRRGFGRRR

SEQ ID NO: 14 - protein, 12 aa, synthetic construct
GRKKRRQRRRPQ

SEQ ID NO: 15 - protein, 7 aa, synthetic construct
PKKKRKV

SEQ ID NO: 16 - protein, 23 aa, synthetic construct
GLFHAIAHFIHGGWHGLIHGWYG

SEQ ID NO: 17 - protein, 30 aa, synthetic construct
WEAALAEALAEALAEHLAEALAEALEALAA

SEQ ID NO: 18 - protein, 10 aa, synthetic construct
HHHHHHHHHH

SEQ ID NO: 19 - protein, 66 aa, synthetic construct
GLFHAIAHFIHGGWHGLIHGWYGWSQPPKKKRKVATPKKSTKKTPKKAKKATPKKSTKKTPKKAKK

SEQ ID NO: 20 - protein, 42 aa, synthetic construct
HHHHHHHHHHATPKKSTKKTPKKAKKATPKKSTKKTPKKAKK

SEQ ID NO: 21 - protein, 56 aa, synthetic construct
GLFHAIAHFIHGGWHGLIHGWYGWSQPPKKKRKVGRKKRRQRRRPQWRRRGFGRRR

SEQ ID NO: 22 - DNA, 45 bp, synthetic construct
  source 1..45: mol_type = other DNA
cctcagcttgtactctagttgttacctctaatgctggacctcagc

SEQ ID NO: 23 - DNA, 45 bp, synthetic construct
  source 1..45: mol_type = other DNA
cctcagcaccctacagagtaacctagattgatcaaacacctcagc

SEQ ID NO: 24 - protein, 19 aa, synthetic construct
KKKKKKKKKKKKKKKKKKK

SEQ ID NO: 25 - protein, 39 aa, synthetic construct
KKKKKKKKKKKKKKKKKKKKKKKKKKKKKKKKKKKKKKK

SEQ ID NO: 26 - protein, 114 aa, synthetic construct
KKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKK

SEQ ID NO: 27 - protein, 5 aa, synthetic construct
  REPEAT 1..5: note = The sequence may be present 1, 2, 3, 4, or 5 times
GGGGA

SEQ ID NO: 28 - protein, 7 aa, synthetic construct
HHHHHHC

SEQ ID NO: 29 - protein, 6 aa, synthetic construct
GGGGGG

(19) United States
(12) Patent Application Publication
(10) Pub. No.: US 2023/0338557 A1
(43) Pub. Date: Oct. 26, 2023

(54) ENHANCED DNA DENDRIMERS AND METHODS OF USE THEREOF

(71) Applicant: Code Biotherapeutics, Inc., Hatfield, PA (US)

(72) Inventors: Robert C. Getts, Hatfield, PA (US); Jennifer Luke, Hatfield, PA (US)

(21) Appl. No.: 18/166,688

(22) Filed: Feb. 9, 2023

Related U.S. Application Data
(60) Provisional application No. 63/308,776, filed on Feb. 10, 2022.

(51) Int. Cl. A61K 47/64; C08G 83/00

(52) U.S. Cl. CPC A61K 47/6455; C08G 83/003

(57) ABSTRACT

The embodiments provide compositions comprising DNA dendrimers, targeting molecules, therapeutic agents, adaptor molecules, support molecules, or combinations thereof. Pharmaceutical compositions, kits, and methods using and producing the same are also provided.

### CROSS-REFERENCE TO RELATED APPLICATIONS

This application claims priority to U.S. Provisional Application No. 63/308,776, filed on Feb. 10, 2022, which is hereby incorporated by reference in its entirety.

### REFERENCE TO SEQUENCE LISTING SUBMITTED ELECTRONICALLY

The instant application contains a sequence listing which has been submitted electronically in XML format and is hereby incorporated by reference in its entirety. Said XML copy, created on 23 May 2023, is named COB-006US_SL.xml and is 30,329 bytes in size.

### FIELD

The present embodiments relate to DNA dendrimers and other molecules linked to, or encoding, targeting molecules, cargo molecules, adaptor molecules, support molecules, or combinations thereof, and methods of using and producing the same.

### BACKGROUND

In many research, diagnostic, and therapeutic applications, the targets of action of a molecule, such as a reporter probe, a small molecule drug, a peptide or a nucleic acid, are intracellular. For nucleic acids that require transcription and/or translation inside a target cell, the nucleic acid must be transported safety into the cell nucleus. However, targeting of a molecule to selected cell types, achieving intracellular delivery to the cytosol and subsequent trafficking to the desired intracellular compartments such as the nucleus represent a challenging goal. Further, therapeutic nucleic acid cargo must be kept safe from degradation, and must express in the nucleus in quantities that are neither too limited or too much. Thus, there is a need for compositions that can reliably deliver molecule such as therapeutic nucleic acids to the nucleus of a target cell without degradation, and with the ability to optimize expression. The present disclosure addresses this need and others.

### SUMMARY

Provided for herein are compositions comprising a DNA dendrimer linked to a targeting moiety and a cargo polynucleotide. In some embodiments, the cargo polynucleotide is linked directly to the DNA dendrimer. In some embodiments, the cargo polynucleotide is linked to the DNA dendrimer by an adaptor molecule. In some embodiments, the cargo polynucleotide comprises at least one promoter and at least one coding sequence encoding for at least one molecule of interest, and has a topology selected from the group consisting of a full circular polynucleotide, a nicked circular polynucleotide, a linear polynucleotide with a closed 5′ and 3′ end, a linear polynucleotide with open 5′ and 3′ ends, and a linear polynucleotide with one open and one closed end. In some embodiments, the cargo polynucleotides herein optionally comprise one or more of a DNA target sequence (DTS), a nuclear localization signal sequence (NLS), or both.

In some embodiments, the cargo polynucleotide comprises a DNA dendrimer binding sequence (DBS). In some embodiments, the DBS links the cargo polynucleotide to the DNA dendrimer. In some embodiments, the adaptor molecule comprises a DBS and a cargo binding region. In some embodiments, the DBS links the adaptor molecule to the DNA dendrimer. In some embodiments, the cargo binding region links the adaptor molecule to the cargo polynucleotide. In some embodiments, the adaptor molecule further comprises one or more of a purification region, a DTS, a NLS, a spacer, a cell penetrating peptide (CPP) sequence, a cleavage site, a flexible linker, or some combination thereof.

In some embodiments, any composition provided for herein may also comprise a support molecule. In some embodiments, the support molecule can associate with the DNA dendrimer. In some embodiments, the support molecule link to the DNA dendrimer. In some embodiments, the support molecule condenses the size of the cargo polynucleotide, the DNA dendrimer, or both.

Also provided for herein are compositions comprising a cargo polynucleotide, a support molecule, and a DNA dendrimer linked to a targeting moiety, wherein the cargo polynucleotide comprises at least one promoter and at least one coding sequence encoding for at least one molecule of interest, and has a topology selected from the group consisting of a full circular nucleotide, nicked circular nucleotide, a linear nucleotide with a closed 5′ and 3′ end, a linear nucleotide with open 5′ and 3′ ends, and a linear nucleotide with one open and one closed end. In some embodiments, the cargo polynucleotide as a full circular topology and is not linked to the DNA dendrimer.

Also provided for herein are plasmids comprising a plasmid backbone comprising at least two restriction sites, at least one promoter, and at least one coding sequence encoding for at least one molecule of interest, wherein the plasmid is capable of forming a cargo polynucleotide with various topologies. In some embodiments, the various topologies are selected from the group consisting of a full circular nucleotide, a nicked circular nucleotide, a linear nucleotide with a closed 5′ and 3′ end, a linear nucleotide with open 5′ and 3′ ends, and a linear nucleotide with one open and one closed end.

In some embodiments, method of delivering a molecule of interest into the nucleus of a target cell are provided, the method comprising contacting the target cell with any composition described herein, wherein the targeting moiety binds to the target cell to allow the composition to enter into the target cell, and wherein the cargo polynucleotide is able to enter into the nucleus of the target cell. In some embodiments, a method of treating a disease is provided, the method comprising administering any composition described herein to a subject to treat the disease, wherein the targeting moiety binds to the target cell to allow the composition to enter into the target cell, and wherein the cargo polynucleotide is able to enter into the nucleus of the target cell. In some embodiments, a method of manufacturing a cargo polynucleotide is provided, the method comprising adding at least one promoter and at least one coding sequence encoding for at least one molecule of interest to a plasmid backbone to form any plasmid described herein, then optionally contacting the plasmid with one or more restriction enzymes.

### BRIEF DESCRIPTION OF THE FIGURES

FIG. 1 depicts a diagram showing a plasmid map, and individual topologies the plasmid can be changed into by contacting or not contacting certain restriction enzymes with the plasmid.

FIG. 2 depicts GFP fluorescence percentages for various polynucleotide topologies.

FIG. 3 depicts a graph showing the number of GFP positive cells per unit area for various polynucleotide topologies.

FIG. 4 depicts relative mean fluorescence of a polynucleotide containing a DNA targeting sequence (DTS) compared to a polynucleotide without a DTS in CHO-K1 cells.

FIG. 5 depicts relative mean fluorescence of a polynucleotide containing a DNA targeting sequence (DTS) compared to a polynucleotide without a DTS in A427 cells.

FIG. 6 depicts relative mean fluorescence of a polynucleotide containing a DNA targeting sequence (DTS) compared to a polynucleotide without a DTS in C2C12 cells.

FIG. 7 depicts relative integral fluorescence of a polynucleotide containing a DNA targeting sequence (DTS) compared to a polynucleotide without a DTS in C2C12 cells.

FIG. 8 depicts relative peak fluorescence of a polynucleotide containing a DNA targeting sequence (DTS) compared to a polynucleotide without a DTS in C2C12 cells.

### DETAILED DESCRIPTION

Unless otherwise defined, all technical and scientific terms used herein have the same meaning as commonly understood by one of ordinary skill in the art. Although methods and materials similar or equivalent to those described herein can be used in the practice or testing, suitable methods and materials are described herein. In the case of conflict, the present specification, including definitions, will control. In addition, the materials, methods, and examples are illustrative only and not intended to be limiting. Other features and advantages of the embodiments provided for herein will be apparent from the present detailed description and claims.

The techniques and procedures for recombinant manipulations, including nucleic acid and peptide synthesis, are generally performed according to conventional methods in the art and various general references (e.g., Sambrook et al, 2001, Molecular Cloning, A Laboratory Approach, Cold Spring Harbor Laboratory Press, Cold Spring Harbor, N.Y.; Ausubel et al., eds, 2005, Current Protocols in Molecular Biology, John Wiley & Sons, New York, N.Y.; and Gerhardt et al., eds., 1994, Methods for General and Molecular Bacteriology, American Society for Microbiology, Washington, D.C.), which are provided throughout this document.

The term “about” or “approximately” means plus or minus 10% of the numerical value of the number with which it is being used. Therefore, about 50% means in the range of 45% 55%. The singular forms “a”, “an”, and “the” include plural reference unless the context clearly dictates otherwise.

The terms “comprise,” “have,” and “include” and their conjugates, as used herein, mean “including but not limited to.” While various compositions, methods, and devices are described in terms of “comprising” various components or steps (interpreted as meaning “including, but not limited to”), the compositions, methods, and devices can also “consist essentially of” or “consist of” the various components and steps.

The term “antibody,” as used herein, refers to an immunoglobulin molecule which can specifically bind to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)2, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, preferably at least about 60% and more preferably at least about 80% of corresponding positions in each of the molecules are occupied by nucleotides which normally base pair with each other (e.g., A:T and G:C nucleotide pairs).

As used herein, a “DNA-based carrier” refers to a delivery system that comprises a deoxyribonucleic acid molecule. A non-limiting example of a DNA-based carrier is a DNA dendrimer. Other DNA-based carriers include double-stranded DNA, single-stranded DNA, and single-stranded hairpin DNA, or multimers thereof.

As used herein, a “DNA dendrimer” or “dendrimer” refers to a matrix of polynucleotides, exhibiting branching, formed by the sequential or generational addition of branched layers to or from a core molecule, such as an initiating monomer.

As used herein, an “initiating monomer” is a polynucleotide compound that serves to nucleate the formation of a dendrimer.

As used herein, an “extending monomer” is a polynucleotide compound that can bind to the initiating monomer and/or to each other during assembly of a dendrimer. Extending monomers form the layers of the dendrimer. The first layer of a dendrimer is the layer of extending monomers closest to the initiating monomer. The outer layer is the layer furthest from the initiating monomer and forming the surface of the dendrimer. Extending monomers are also referred to in the art as matrix monomers, matrix extending monomers and matrix polynucleotide monomers.

The terms “substituting,” “substituted,” “mutating,” or “mutated” as used herein refer to altering, deleting, or inserting one or more amino acids or nucleotides in a polypeptide or polynucleotide sequence to generate a variant of that sequence.

The terms “polynucleotide” or “nucleic acid molecule” means a molecule comprising a chain of nucleotides covalently linked by a sugar-phosphate backbone or other equivalent covalent chemistry. Double and single-stranded DNAs and RNAs are non-limiting examples of polynucleotides.

The term “polypeptide” or “protein” means a molecule that comprises at least two amino acid resides linked by a peptide bond to form a polypeptide. In some embodiments, the term “peptide” can also be used.

The term “variant” as used herein refers to a polypeptide or polynucleotide that differs from a reference polypeptide or a reference polynucleotide by one or more modifications, including substitutions, insertions, or deletions.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

As used herein, the term “promoter/regulatory sequence” means a nucleic acid sequence which controls or facilitates the expression of a gene product operably linked to the promoter/regulator sequence. In some instances, this sequence may be the core promoter sequence and in other instances, this sequence may also include an enhancer sequence and other regulatory elements which are control or facilitate the expression of the gene product. The promoter/regulatory sequence may, for example, be one which expresses the gene product in an inducible manner. An “inducible” promoter is a nucleotide sequence which, when operably linked with a polynucleotide that encodes or specifies a gene product, causes the gene product to be produced substantially only when an inducer which corresponds (e.g., binds) to the promoter is present.

As used herein, a “targeting moiety” refers to a molecule that binds to a molecule present on the cell surface of a target cell.

As used herein, a “targeted DNA-based carrier” and “a DNA-based carrier comprises a targeting moiety” refers to a composition comprising a DNA-based carrier and a targeting moiety. The targeting moiety may be linked directly, or by means of a linker, to the DNA-based carrier. Alternatively, the targeting moiety may be linked to another molecule, such as a cargo molecule or a secondary, non-nucleic acid carrier in the composition. The DNA-based carrier is targeted by virtue of being present in the same composition with the targeted cargo or targeted secondary carrier.

The term “vector” as used herein refers to a nucleic acid which can infect, transfect, transiently or permanently transduce a cell. It will be recognized that a vector can be a naked nucleic acid, or a nucleic acid complexed with protein or lipid. Vectors include, but are not limited to, replicons (such as RNA replicons, bacteriophages) to which fragments of DNA may be attached and become replicated. Vectors thus include, but are not limited to RNA, autonomous self-replicating circular or linear DNA or RNA (i.e., “plasmids”), and include both the expression and non-expression plasmids. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure or is incorporated within the host's genome.

“Promoter” as used herein means a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. Such promoter-enhancers can modify, for example, but not limited to, tissue specificity or transduction efficiency. A promoter can be derived from sources including viral, bacterial, fungal, plants, insects, and animals. A promoter can regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents.

The term “topology” as used herein refers to different structural arrangement of any vector, plasmid, or polynucleotide disclosed herein. For example, polynucleotides can have a circular topology, such as a plasmid, where the polynucleotide has no 5′ or 3′ end. Polynucleotides disclosure herein can also have linear topologies, where the polynucleotide has a 5′ and 3′ end. Polynucleotides with a liner topology may have one or both ends of the polynucleotide covalently closed. In some embodiments, certain restriction enzymes can covalently close a DNA end, for example, telomerase N (Tel N).

### Compositions

### A. DNA-Based Carriers

In some embodiments, compositions disclosed herein comprise a DNA-based carrier. DNA-based carriers include, but are not limited to, DNA dendrimers, double-stranded linear DNA, single-stranded linear DNA, and single-stranded hairpin DNA, formulated either as monomolecular structures or including several units of the conformation cross-linked together (e.g., multimers). In some embodiments, the composition comprises a DNA dendrimer. DNA dendrimers are spheroid particles (diameter typically about 130 to about 150 nm, although they can be designed to be other sizes) of flexible branches formed by inter-hybridized DNA monomers. Each DNA monomer is composed of two polynucleotide strands that share a central region of complementary sequences where the two strands hybridize to each other, leaving 4 terminal single-stranded polynucleotide portions. These terminal sequences are complementary among themselves; hence, they can hybridize (in layers) to terminal sequences of other DNA monomers. Optionally, DNA dendrimers comprise covalently cross-linked strands of DNA.

DNA dendrimers are commercially available. In addition, the structural design and assembly of DNA dendrimers is generally known in the art. See, for instance, U.S. Pat. Nos. 5,175,270 and 6,274,723, each of which are incorporated herein by reference in their entireties. An initiating monomer constitutes the approximate center of a dendrimer, depending on the type of branching in the dendrimer. The three-dimensional assembly of extending monomers around the initiating monomer forms the interior volume of the dendrimer. The last, outer layer of extending monomers forms the surface of the dendrimer. Thus, the assembly of a dendrimer results in a three-dimensional shape, typically, but not exclusively, a roughly spherical shape comprising layers of extending monomers. The outer layer comprises numerous binding sites.

More specifically, DNA dendrimers may be prepared by protocols having the following features. (i) The starting material is a double-stranded duplex of DNA with 5′ and 3′ single-stranded overhangs, or “binding arms,” attached to the duplex trunk (e.g., four binding arms total), called the “initiating monomer,” which is descriptive of its role in the assembly of a dendrimer. Each initiating monomer's 5′ and 3′ binding arms are annealed to complementary binding arms on “extending monomers” that have similar composition and morphology. (ii) A subset of the four binding arms on each extending monomer is complementary to the binding arms on the initiating monomer. The non-complementary binding arms of the extending monomers are inactive for annealing to the initiating monomer. Typically, four extending monomers can anneal to the initiating monomer to yield a single-layer, or one-layer, dendrimer in solution. (iii) To add another layer of extending monomers to dendrimers, one typically adds similar but distinguishable extending monomers, in which each monomer has a subset of its four binding arms that is complementary to binding arms on the dendrimer. Thus, a one-layer dendrimer can be converted to a two-layer dendrimer, and so on, stepwise, until a desired size of dendrimer is reached. Typically, dendrimers of three or four layers are used.

After assembly, a DNA dendrimer can be crosslinked to maintain and stabilize the structure of the dendrimer. Crosslinking hybridized regions between monomers (i.e., inter-monomer crosslinking) or between monomers and the nucleic acids that carry detectable labels, as well as between trunk portions (intra-monomer crosslinking), can stabilize the structure of the polynucleotide dendrimer. Similarly, any hybridized region of any DNA-based carrier may be crosslinked to stabilize the carrier. Individual units of carrier may also be crosslinked into a polymolecular carrier. Such crosslinking chemistries are well known in the art. See, e.g., Cimino et al., Annu. Rev. Biochem. 54:1151-1193 (1985); Shi et al., Biochemistry 25:5895-5902 (1986); and Cimino et al., Biochemistry 25:3013-3020 (1986). See also U.S. Pat. No. 4,196,281. Non-limiting examples of suitable crosslinking agents include: psoralens (including but not limited to 8-methoxypsoralen and angelicin), mitomycin C, daunomycin, ethidium diazide, cisplatin, transplatin, carboplatin, 8-methoxypsoralen, mechlorethamine, oxaliplatin, and carbodiimide compounds, among others.

The polynucleotide strands used in the monomers of dendrimers or in the other DNA-based carriers, can be made using standard techniques for synthesis of nucleic acids. These techniques can be biological or chemical. The techniques and procedures are generally performed according to conventional methods in the art and in various general references (e.g., Sambrook et al., 2001, supra; Ausubel et al., eds., 2005, supra, and Gerhardt et al., eds., 1994, Methods for General and Molecular Bacteriology, American Society for Microbiology, Washington, D.C.).

In some embodiments, polynucleotides are chemically synthesized using methods known in the art. See, e.g., Gait, 1985, Oligonucleotide Synthesis: A Practical Approach (IRL Press, Oxford, England. In another embodiment, polynucleotides are synthesized enzymatically using the polymerase chain reaction (PCR). One PCR method suitable for generating single-stranded polynucleotides is multi-cycle PCR using a single primer, which thereby amplifies a single strand. Nucleic acids may be purified by any suitable means, as are well known in the art, prior to their use. For example, the nucleic acids can be purified by reverse phase or ion exchange HPLC, size exclusion chromatography or gel electrophoresis. The skilled artisan will recognize that the method of purification will depend in part on the size of the nucleic acid to be purified.

The outer layer of a DNA dendrimer can have at least two types of binding arms. These binding arms can be used for attaching one or more types of moieties. In some embodiments, the DNA dendrimer of the compositions can bind or attach to targeting moieties, cargoes, support molecules, and the like.

In some embodiments, the DNA-based carrier is associated with a secondary carrier, which may serve as a scaffold for the DNA-based carriers. Examples of such secondary carriers include, but are not limited to, liposomes, non-DNA dendrimers, polymer carriers, microbubbles, paramagnetic and ferromagnetic particles, self-assembled polymers, polymersomes, filomicelles, albumin particles, lipoproteins, and the like. A self-assembled polymer is one that is formed by self-assembly of monomolecular building blocks. These building blocks can be amphiphilic copolymers, which comprise a hydrophilic component (such as, but not limited to, polyethylenimine or polyethyleneglycol) and a hydrophobic component (such a, but not limited to, aliphatic polyesters) into a core-shell-type structure. These structures that are not maintained by direct conjugation or crosslink of the building blocks are known to as micelles and can vary in their morphology, for instance from spherical micelles (“polymersomes”) to elongated or filamentous rods (“filomicelles”). The sizes of these structures may also vary from the nanometer to the micrometer size range. Once formed by self-assembly, these structures may alternatively be further crosslinked chemically to increase their stability. Hydrophobic cargoes can be embedded into the hydrophobic regions of the carrier, while hydrophilic cargoes can be incorporated into an internal aqueous core or to the external hydrophilic corona.

In some embodiments, the DNA-based carrier is linked to the surface of the secondary carrier. In some embodiments, the DNA-based carrier is not linked but is associated with the secondary carrier by virtue of being in the same composition. In some embodiments, the cargos and targeting moieties can be linked to the DNA-based carrier, or to the scaffold carrier, using methods described herein and known in the art.

### B. Targeting Moieties

In some embodiments, the DNA-based carrier is directed to a specific cell by linking a targeting moiety to the carrier, to a secondary carrier and/or a cargo. In some embodiments, the targeting moiety is linked to the DNA-based carrier. In some embodiments, the targeting moiety is linked to a secondary carrier. A targeting moiety may be an antibody, a naturally-occurring ligand for the receptor or a functional derivative thereof, a vitamin, a hormone, a small molecule mimetic of a naturally-occurring ligand, a peptide, a polypeptide, a peptidomimetic, a carbohydrate, a lipid, a FN3 domain, an aptamer, a nucleic acid, a toxin, a component of a microorganism, or any other molecule provided it binds specifically to the cell surface molecule and induces endocytosis of the bound moiety.

Without being bound to any particular theory, the targeting moiety can bind specifically to a molecule on the cell surface of a target cell. The targeting moiety can be bind to a cell surface molecule, which can, in some embodiments, induce endocytosis of the DNA-based carrier. Non-limiting examples of cell surface molecule that may be targeted include cell surface proteins, carbohydrates, and lipids. Cell surface molecules that may be targeted include molecules associated with classical endocytosis and those associated with non-classical endocytosis.

In some embodiments, the target cell surface molecule is a cell adhesion molecule (CAM). Cell adhesion molecules useful in the invention include, but are not limited to, neural specific adhesion molecules (e.g., NCAM) and systemic intercellular adhesion molecules. Systemic CAMs include intercellular adhesion molecules (e.g., ICAM-1, ICAM-2, ICAM-3), platelet-endothelial cell adhesion molecule (PECAM), activated leukocyte cell adhesion molecule (ALCAM), B-lymphocyte cell adhesion molecule (BL-CAM), vascular cell adhesion molecule (VCAM), mucosal vascular addressin cell adhesion molecule (MAdCAM), CD44, LFA-2 (CD2), LFA-3 (CD58), basigin (CD147) and the like. In some embodiments, the cell surface molecule is CD71.

In some embodiments, the targeting moiety is an antibody that specifically binds to a target cell surface molecule. In some embodiments, the antibody is selected from the group consisting of a polyclonal antibody, a monoclonal antibody, a humanized antibody, a synthetic antibody, a heavy chain antibody, and a biologically active fragment of an antibody, wherein the biologically active fragment is a Fab fragment, a F(ab′)2 fragment, an sc-Fv fragment.

When the antibody used as a targeting moiety in the compositions and methods of the invention is a polyclonal antibody (IgG), the antibody is generated by inoculating a suitable animal with the targeted cell surface molecule. Antibodies produced in the inoculated animal which specifically bind to the cell surface molecule are then isolated from fluid obtained from the animal. Antibodies may be generated in this manner in several non-human mammals such as, but not limited to goat, sheep, horse, camel, rabbit, and donkey. Methods for generating polyclonal antibodies are well known in the art and are described, for example in Harlow, et al. (1988, In: Antibodies, A Laboratory Manual, Cold Spring Harbor, N.Y.).

Monoclonal antibodies directed against a full-length targeted cell surface molecule or fragments thereof may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Human monoclonal antibodies may be prepared by the method described in U.S. patent publication 2003/0224490. Monoclonal antibodies directed against an antigen are generated from mice immunized with the antigen using standard procedures as referenced herein. Nucleic acid encoding the monoclonal antibody obtained using the procedures described herein may be cloned and sequenced using technology available in the art, and is described, for example, in Wright et al. (1992, Critical Rev. in Immunol. 12(3,4):125-168) and the references cited therein.

To prepare a targeted DNA-based carrier, such as a DNA dendrimer, the targeting moiety can be linked to the DNA-based carrier, or a secondary carrier or a cargo that is linked to, or associated with, a composition comprising the DNA-based carrier. A single targeting moiety may be linked to the DNA-based carrier or secondary carrier. Alternatively, a plurality of (e.g., two or more) targeting moieties are linked to the DNA-based carrier, secondary carrier or cargo. When a plurality of targeting moieties are linked to a carrier or cargo, the moieties may target the same cell surface molecule or may target different cell surface molecules. If targeting different cell surface molecules, these molecules may be associated with the same endocytic pathway or different endocytic pathways. The targeting moiety may also not be associated with an endocytic pathway. If targeting different cell surface molecules, the cell surface molecules may be present on the same cell type or may be present on different cell types.

Linking may be non-covalent or covalent. A targeting moiety may be linked directly to one or more of the polynucleotide strands comprising the DNA-based carrier. Alternatively, a targeting moiety is linked to a linker molecule which is in turn linked to the DNA-based carrier. In some embodiments, wherein the DNA-based carrier is a DNA dendrimer, the linker molecule is an oligonucleotide comprising a sequence substantially complementary to a sequence present in one of the binding arms on the surface of the DNA dendrimer. Thus, the targeting moiety is linked indirectly and non-covalently to the DNA dendrimer by hybridization of the oligonucleotide to a binding arm. This approach is also applicable to other DNA-based carriers. Optionally, the hybridized oligonucleotide is also cross-linked to the DNA dendrimer. Cross-linking chemistries are disclosed elsewhere herein. When not cross-linked, hybridization between the DNA dendrimer and an oligonucleotide linked to a targeting moiety should be sufficiently long-lived under the conditions of use. Determination of binding free energies for nucleic acid molecules is well known in the art (see, e.g., Turner et al., 1987, CSH Symp. Quant. Biol. LII pp. 123-133; Freier et al., 1986, Proc. Nat. Acad. Sci. USA 83:9373-9377; Turner et al., 1987, J. Am. Chem. Soc. 109:3783-3785; Chavali et al., 2005, Bioinformatics 21(20):3918-3925).

In some embodiments, the linker is a secondary IgG Fc-specific antibody which is linked to the DNA-based carrier. In some embodiments, the antibody specifically binds to the Fc portion of the primary IgG antibody, e.g., the targeting moiety or an intervening antibody. The secondary antibody is preferably specific for the species source of the primary antibody. For instance, if the targeting moiety is a human IgG antibody, the secondary antibody is anti-human IgG. In another aspect, the secondary antibody recognizes epitopes of the entire primary antibody, not just the Fc portion. Alternatively, the linker is an Fe gamma receptor that binds specifically to the Fc portion of an IgG antibody. In any of these, the carrier may be readily linked to any targeting moiety that is an IgG antibody.

Non-covalent linkages include, but are not limited to, affinity binding pairs, such as biotin-streptavidin and immunoaffinity, having sufficiently high affinity to maintain the linkage during use and are well-known in the art. The art is also replete with conjugation chemistries useful for covalently linking a target moiety to a DNA-based carrier, secondary carrier or cargo, directly or via a linker. Art-recognized covalent coupling techniques are disclosed, for instance, in U.S. Pat. Nos. 5,416,016, 6,335,435, 6,528,631, 6,861,514 and 6,919,439, incorporated herein by reference in their entirety. Other conjugation chemistries are disclosed in U.S. Patent Publication No. 20040249178, incorporated herein by reference in its entirety. Still other conjugation chemistries include: p-hydroxy-benzoic acid linkers (Chang-Po et al., 2002, Bioconjugate Chem. 13(3):525-529); native ligation (Stetsenko et al., 2000, J Org. Chem. 65:4900-4908): disulfide bridge conjugates (Oehlke et al., 2002, Eur J. Biochem. 269:4025-4032 and Rogers et al., 2004, Nuc Acids Res. 32(22) 6595-6604); maleimide linkers (Zhu et al., 1993, Antisense Res Dev. 3:265-275); thioester linkers (Ede et al., 1994, Bioconjug Chem. 5:373-378); Diels-Alder cycloaddition (Marchin et al., 2006. Nuc Acids Res. 34(3): e24, 2006 Feb. 14 Epub); U.S. Pat. No. 6,656,730 and the like. For reviews of peptide-oligonucleotide conjugation chemistries, see also Tung et al., 2000, Bioconjugate Chem. 11:605-618; Zatsepin et al., 2005, Curr Pharm Des. 11(28):3639-3654; and Juliano, 2005, Curr Opin Mol. Ther. 7(2):132-136.

### C. Cargos

The compositions provided herein can also comprise a cargo. In some embodiments, the cargo is attached directly or indirectly to a DNA dendrimer. A cargo that is attached to the DNA dendrimer is associated with the DNA dendrimer via a linker or other type of molecule (adaptor), and the like. One, two, or more different cargoes may be delivered by a targeted DNA-based carrier. Cargoes that can be delivered in accordance the compositions disclosed herein include, but are not limited to, a variety of agents, including, but not limited to, therapeutic agents, imaging agents, monitoring agents, chemotherapeutic agents, anti-oncogenic agents, anti-angiogenic agents, tumor suppressor agents, anti-microbial agents, enzyme replacement agents, gene expression modulating agents and expression constructs comprising a nucleic acid encoding a therapeutic protein or nucleic acid. In some embodiments, cargo may be an exogenous material or an endogenous material. Cargoes include any molecule that induces an effect in a cell, including any protein, nucleic acid, small molecule, carbohydrate, or lipid. Cargoes may be peptides, proteins (including enzymes, antibodies and peptidic hormones), ligands of cytoskeleton, nucleic acid, small molecules, non-peptidic hormones and the like. Nucleic acids and cargo polynucleotides that may be delivered by the method of the invention include synthetic and natural nucleic acid material, including DNA, RNA, transposon DNA, antisense nucleic acids, dsRNA, siRNAs, transcription RNA, messenger RNA, ribosomal RNA, small nucleolar RNA, microRNA, ribozymes, plasmids, and expression constructs. When the cargo is or comprises a nucleic acid, the nucleic acid may be a separate entity from the DNA-based carrier. In these embodiments, the DNA-based carrier is not itself the cargo.

In some embodiments, the cargo is a polynucleotide comprising at least one promoter and at least one coding sequence that encodes for at least one molecule of interest. For example, the cargo polynucleotide may comprise one promoter and two or more coding sequences that encode for different molecules of interest, or one promoter for each coding sequence present. In some embodiments, the promoter or promoters are tissue specific promoters that transcript or translate the at least one molecule of interest only when the composition comprising the cargo polynucleotide is present within a target cell that matches the tissue specific promoter.

In some embodiments, the molecule of interest is a gene product, such as an enzyme, protein, receptor, and the like. In some embodiments, the cargo polynucleotide encodes two or more molecules of interest. In some embodiments, at least one of the molecules of interest is a detectable molecule, such as, but not limited to, proteins are tagged, detectable via bioluminescence, fluorescence, radiation signals, or some combination thereof. In some embodiments, such molecules of interest are useful for biodistribution studies and other quantitative and/or real-time assessments.

In some embodiments, the cargo polynucleotide may have one or more structures or topologies. For example, the cargo polynucleotide may exist as a fully circular, double stranded DNA or RNA with no free 5′ or 3′ ends. In some embodiments, the cargo polynucleotide is a plasmid. In some embodiments, the cargo polynucleotide can be a nicked circular polynucleotide, a linear polynucleotide with a closed 5′ and 3′ end, a linear polynucleotide with open 5′ and 3′ ends, and a linear polynucleotide with one open and one closed end. In some embodiments, a nicked circular polynucleotide is a fully circular, double stranded DNA or RNA that has at least one break in a single strand of the DNA or RNA. In some embodiments, the nick can be due to the action of a nicking restriction enzyme. In some embodiments, a linear polynucleotide has defined 5′ and 3′ ends, with either end, or both, being optionally closed via a covalent bond.

The different topologies or structures can be used for different attachments to the DNA dendrimer.

Provided herein is a plasmid comprising a plasmid backbone comprising at least two restriction sites, at least one promoter, and at least one coding sequence encoding for at least one molecule of interest, wherein the plasmid can form a cargo polynucleotide with various topologies, such as those provided for herein. For example, a gene cassette comprising a promoter sequence and a coding sequence encoding for at least one molecule of interest can be inserted into the plasmid backbone. The resulting plasmid can have a fully circular topology, and if such a topology is desired, no further steps are required. However, if a different topology is desired, the plasmid can be contacted with one or more restriction enzymes to break one or both nucleotide strands of the plasmid and create a nicked circular polynucleotide, a linear polynucleotide with a closed 5′ and 3′ end, a linear polynucleotide with open 5′ and 3′ ends, or a linear polynucleotide with one open and one closed end. In some embodiments, closed ends on liner polynucleotides are formed using telomerase N (Tel N).

In some embodiments, cargo polynucleotide is transported to the nucleus of the cell. In some embodiments, the cargo polynucleotide can also comprise one or more of a DNA targeting sequence (DTS) or a nuclear localization signal (NLS). DTS and NLS are, independently, short amino acid sequences that target proteins and associated nucleic acids for import into the nucleus. For example, an NLS that can be encoded by the cargo polynucleotide has the amino acid sequence of YPDEVKRKKKP (SEQ ID NO: 1) or SLLESPFDKPDEVKRKKKPPTSHQSDATAEDDSSSKKK (SEQ ID NO: 2). These are non-limiting examples of NLS sequences and any NLS can be used. In some embodiments, at least one DTS, at least one NLS, or both can be located anywhere within or in respect to the cargo polynucleotide. For example, the DTS, NLS, or both can be located upstream or downstream of the at least one promoter of the cargo polynucleotide. In such a molecule, the polypeptide is linked to the cargo polynucleotide.

In some embodiments, one or more DTS or NLS sequences can be attached or linked to the cargo polynucleotide, instead of being located within the cargo polynucleotide. In some embodiments, the one or more DTS or NLS sequences can be included in any adaptor molecule described herein, wherein the adaptor molecule is linked to the cargo polynucleotide. In some embodiments, the one or more DTS or NLS sequences can be linked directly to the cargo polynucleotide without being associated with a corresponding adaptor molecule. In some embodiments, the DTS or NLS sequence can be attached to a cargo polynucleotide via a modified nucleotide. For example, a DTS or NLS sequence can be attached to a nicked circular cargo polynucleotide at the nick site, optionally where a modified nucleotide has been first inserted at the nick site.

In some embodiments, the cargo, including cargo polynucleotides described herein, can be linked to a DNA dendrimer directly by any covalent or non-covalent interaction or bond described herein, such as a hydrogen bond. Thus, in some embodiments, the cargo polynucleotide comprises a DNA dendrimer binding sequence (DBS), which links the cargo polynucleotide to the DNA dendrimer. In some embodiments, the DBS comprises a nucleic acid sequence that is complimentary to a nucleic acid sequence on the DNA dendrimer. For example, the DBS comprises a nucleic acid sequence that is complimentary to at least one binding arm of the DNA dendrimer. In some embodiments, the DBS binds to the DNA dendrimer via hydrogen bonding. In some embodiments, the DBS comprises a nucleic acid sequence of TAGAGGTAACAACTAGCGTACAA (SEQ ID NO: 3). In some embodiments, the DBS sequence further comprises a polythymine sequence at either the 5′ or 3′ end of the sequence.

For example, in some embodiments, the DBS comprises a nucleic acid sequence of TAGAGGTAACAACTAGCGTACAATTTTTTTTTT (SEQ ID NO: 4). In some embodiments, the DBS comprises a nucleic acid sequence of CCTCAGCTTGTACTCTAGTTGTTACCTCTAATGCTGGACCTCAGC (SEQ ID NO: 22). In some embodiments, the DBS comprises a nucleic acid sequence of

(SEQ ID NO: 23)
CCTCAGCACCCTACAGAGTAACC
TAGATTGATCAAACACCTCAGC.

These are non-limiting examples of DBS molecules and any DBS sequence can be used that is complimentary to the dendrimer arm.

### D. Adaptor Molecules

The cargo can also be linked to a DNA dendrimer by an adaptor molecule as provided for herein. In some embodiments, the adaptor molecule comprises DNA dendrimer binding sequence (DBS) that links the adaptor molecule to the DNA dendrimer, and a cargo binding region that links to the cargo, for example, a cargo polynucleotide as provided for herein.

In some embodiments, the DBS comprises a nucleic acid sequence that is complimentary to a nucleic acid sequence on the DNA dendrimer. For example, the DBS comprises a nucleic acid sequence that is complimentary to at least one binding arm of the DNA dendrimer. In some embodiments, the DBS binds to the DNA dendrimer via hydrogen bonding. In some embodiments, the DBS comprises any DBS sequence disclosed herein. In some embodiments, any DBS sequence can be used that is complimentary to the dendrimer arm.

In some embodiments, the cargo binding region can be linked to the cargo polynucleotide by range of linkers known in the art. For example, the cargo binding region can comprise a nucleic acid sequence that is complimentary to at least one part of a cargo polynucleotide, and/or links to the cargo by DNA ligation. In another example, the cargo binding region can link to a cargo polynucleotide by chemical coupling, such as, but not limited to, click chemistry or EDC cross-linking. In some embodiments, the cargo binding region comprises at least one nucleotide with an amine, azide, or other reactive group. In some embodiments, the cargo binding region comprises at least one cysteine residue.

The adaptor molecule can also have other regions or features included. In some embodiments, such regions or features can be located between the DBS and the cargo binding region but may also be located in any order within the adaptor molecule.

In some embodiments, the adaptor molecule further comprises at least one purification region. In some embodiments, purification region comprises at least one purification or affinity tag. The purification or affinity tag can be any such chemical or amino acid tag known in the art, for example, a polyhistidine tag such as, but not limited to, His6, His 12, and the like.

In some embodiments, the adaptor molecule further comprises a DTS, NLS, or both as provided for herein. In some embodiments, the DTS and NLS regions are located between the DBS and the cargo binding region. In some embodiments the NLS has an amino acid sequence of YPDEVKRKKKP (SEQ ID NO: 1) or SLLESPFDKPDEVKRKKKPPTSHQSDATAEDDSSSKKK (SEQ ID NO: 2). In some embodiments the DTS or NLS regions further comprise at least one spacer located either before or after the STS or NLS regions. If two spacers are present, they are located immediately before and after the DTS, NLS, or both. Spacers are generally known in the art and comprise a range of flexible or semi-flexible amino acids or molecules. In some embodiments, the spacer or spacers comprise a polyglycine, optionally with alanine and/or serine residues. In some embodiments, the spacer or spacers have the amino acid sequence of GGGG (SEQ ID NO: 5). In some embodiments, the spacer or spacers comprise polyethylene glycol (PEG), propylene glycol alginate (PGA), PEG-polylactic acid (PLA), poly lactic-co-glycolic acid (PGLA), or any combination thereof. In some embodiments, the spacer or spacers comprise a saturated or unsaturated hydrocarbon chain comprising 3-6 carbons that can optionally be substituted.

In some embodiments, the adaptor molecule further comprises a cell penetrating peptide sequence (CPP). CPPs are a group of short peptides that have the ability to increase membrane transduction and can be used to assist the transportation of molecule through cell membrane (for review see, Xu et al., 2019, J Control Release, 309:106-124). A wide range of CPPs may be included in the adaptor molecule to assist the DNA dendrimer-based composition to pass into a target cell or target nucleus. For example, in some embodiments, the CPP has an amino acid sequence of QPRRRPRRKKRG (SEQ ID NO: 6). In some embodiments, the CPP is located between the DBS and the cargo binding region.

In some embodiments, the cargo is separable from the DNA dendrimer. Thus, in some embodiments the adaptor molecule further comprises one or more cleavage sites. Cleavage sites may be located anywhere between the DBS and the cargo binding region. In some embodiments, the cleavage site is located adjacent to, or nearly adjacent to, the DBS. For example, in some embodiments, the cleavage site is a valine-citruline/p-aminobenzyl carbamate (val-cit) cleavable linker. Val-cit linkers are known in the art and are used in drug conjugation to release cargoes from targeting moieties. Other cleavable linkers known in the art may also be included, such as a ribozyme self-cleaving cleavage site, or tunable pH-sensitive linkers (see, e.g., Choy et al., Bioconjugate Chem. (2016) 27(3):824-830).

In some embodiments, the adaptor molecule further comprises at least one flexible linker. In some embodiments, the flexible linker is located between the DNS and the cargo binding region. In some embodiments, the at least one flexible linker is or comprises polyethylene glycol (PEG), propylene glycol alginate (PGA), PEG-polylactic acid (PLA), poly lactic-co-glycolic acid (PGLA), (GG)n, (GGGGS)n (SEQ ID NO: 7), (GGGGA)n (SEQ ID NO: 27), or any combination thereof, wherein each n is independently, 1-5.

Adaptor molecules provided for herein may have none, some, or all ofthe optional elements disclosure herein. For example, adaptor molecules may comprise one or more purification regions, DTS, NLS, CPP, spacers, cleavage sites, flexible linkers, or any combination thereof, in addition to the DBS and cargo binding region. Non-limiting examples of adaptor molecules are listed below.

| | |
|---|---|
| DBS, purification region, | TAGAGGTAACAACTAGCGTAC |
| cargo binding region | AATTTTTTTTTTHHHHHHC |
| | SEQ ID NO: 8 |
| DBS, flexible linker, NLS, | TAGAGGTAACAACTAGCGTAC |
| cargo binding region | AATTTTTTTTTTGGGGGGYPD |
| | EVKRKKKP SEQ ID NO: 9 |
| DBS, flexible linker, | TAGAGGTAACAACTAGCGTAC |
| cleavage site, spacer, NLS, | AATTTTTTTTTTGGGGGGCit |
| spacer, purification region, | VGGGGYPDEVKRKKKPGGGGH |
| cargo binding region | HHHHHC SEQ ID NO: 10 |
| DBS, flexible linker, | TAGAGGTAACAACTAGCGTAC |
| cleavage site, CPP, NLS, | AATTTTTTTTTTGGGGGGCit |
| spacer, purification region, | VQPRRRPRRKKRGSLLESPFD |
| cargo binding region | KPDEVKRKKKPPTSHQSDATA |
| | EDDSSSKKKGGGGHHHHHHC |
| | SEQ ID NO: 11 |

In some embodiments, a composition provided comprises a targeting moiety and a cargo disclosed herein, wherein the targeting moiety and the cargo are linked by an adaptor molecule comprising at least one DTS. In some embodiments, the targeting moiety/adaptor molecule with DTS/cargo composition does not comprise a DNA dendrimer.

In some embodiments, a composition comprises the sequences provided in the table below. In some embodiments, a composition comprises, from 5′ to 3′ end, the sequences provided in the table below.

| TAGAGGTAACA | HHHHH | | | | | |
|---|---|---|---|---|---|---|
| ACTAGCGTACA | HC (SEQ | | | | | |
| ATTTTTTTTTT | ID NO: | | | | | |
| (SEQ ID NO: 4) | 28) | | | | | |
| TAGAGGTAACA | GGGGG | YPDEVK | | | | |
| ACTAGCGTACA | G (SEQ | RKKKP | | | | |
| ATTTTTTTTTT | ID NO: | (SEQ ID | | | | |
| (SEQ ID NO: 4) | 29) | NO: 1) | | | | |
| TAGAGGTAACA | GGGGG | CitV | GGGG | YPDEVKRK | GGGG | HHHH |
| ACTAGCGTACA | G (SEQ | | (SEQ | KKP | (SEQ ID | HHC |
| ATTTTTTTTTT | ID NO: | | ID NO: | (SEQ ID | NO: 5) | (SEQ |
| (SEQ ID NO: 4) | 29) | | 5) | NO: 1) | | ID NO: |
| | | | | | | 28) |
| TAGAGGTAACA | GGGGG | CitV | QPRR | SLLESPFDK | GGGG | HHHH |
| ACTAGCGTACA | G (SEQ | | RPRR | PDEVKRKK | (SEQ ID | HHC |
| ATTTTTTTTTT | ID NO: | | KKRG | KPPTSHQSD | NO: 5) | (SEQ |
| (SEQ ID NO: 4) | 29) | | (SEQ | ATAEDDSSS | | ID NO: |
| | | | ID NO: | KKK (SEQ | | 28) |
| | | | 6) | ID NO: 2) | | |

In some embodiments, a composition comprises, from 5′ to 3′ end, the sequences of SEQ ID NO: 4 and SEQ ID NO: 28. In some embodiments, a composition comprises, from 5′ to 3′ end, the sequences of SEQ ID NO: 4, SEQ TD NO: 29, and SEQ ID NO: 1. In some embodiments, a composition comprises, from 5′ to 3′ end, the sequences of SEQ ID NO: 4, SEQ ID NO: 29, CitV, SEQ ID NO: 5, SEQ TD NO: 1, SEQ ID NO: 5, and SEQ ID NO: 28. In some embodiments, a composition comprises, from 5′ to 3′ end, the sequences of SEQ ID NO: 4, SEQ ID NO: 29, CitV, SEQ TD NO: 6, SEQ ID NO: 2, SEQ ID NO: 5, and SEQ TD NO: 28.

In some embodiments, a composition comprises, from 5′ to 3′ end, the sequences of SEQ ID NO: 4 and SEQ ID NO: 28, wherein the sequences are conjugated to each other. In some embodiments, a composition comprises, from 5′ to 3′ end, the sequences of SEQ ID NO: 4, SEQ ID NO: 29, and SEQ ID NO: 1, wherein the sequences are conjugated to each other. In some embodiments, a composition comprises, from 5′ to 3′ end, the sequences of SEQ ID NO: 4, SEQ ID NO: 29, CitV, SEQ TD NO: 5, SEQ TD NO: 1, SEQ ID NO: 5, and SEQ ID NO: 28, wherein the sequences are conjugated to each other. In some embodiments, a composition comprises, from 5′ to 3′ end, the sequences of SEQ ID NO: 4, SEQ ID NO: 29, CitV, SEQ ID NO: 6, SEQ ID NO: 2, SEQ ID NO: 5, and SEQ ID NO: 28, wherein the sequences are conjugated to each other.

### E. Support Molecules

The compositions herein may also comprise one or more support molecules. Support molecules as provided for herein can be used for a range of tasks, including, but not limited to, protecting DNA dendrimers and cargo polynucleotides from nuclease degradation, enhancing transfection efficiency, assisting in nuclear delivery, and condensing the size of the DNA dendrimer, cargo polynucleotide, or both. Support molecules can be covalently or non-covalently linked to the DNA dendrimer in the same or similar manner as the targeting moieties and cargos. However, in some embodiments, one or more support molecules are not linked to the DNA dendrimer and can associate with the composition through other means.

Without wishing to be bound by theory, because many polynucleotide structures have a negatively changed amine backbone, a support molecule with a sufficient positive charge can associate with the composition through charge attraction. Thus, in some embodiments, the one or more support molecules associate with the composition through charge attraction. In some embodiments, the one or more support molecules have a net-positive charge that is high enough to allow for the one or more support molecules to interact with and, optionally, compact the negatively charged DNA dendrimer. In some embodiments, the one or more support molecules have a net-positive charge that is low enough to avoid aggregation or other cytotoxic side effects.

Additionally, such support molecules can also associate with cargo polynucleotides and DNA dendrimers simultaneously. Thus, in some embodiments, compositions provided for herein comprise a cargo polynucleotide, a support molecule, and a DNA dendrimer linked to a targeting moiety, wherein the cargo polynucleotide is not covalently linked to the DNA dendrimer. The support molecule can associate the cargo polynucleotide with the DNA dendrimer in this way regardless of the topology of the cargo polynucleotide. However, when the cargo polynucleotide has a full circular topology, without any nicks, sites, or regions for direct covalent linkage to a DNA dendrimer or an adaptor molecule, the one or more support molecule can associate the cargo polynucleotide and the DNA dendrimer together to form the composition.

Support molecules are provided for herein can be used individually, as repeats, or in combination either within one molecular entity or a complex mixture of individual molecular entities. In some embodiments, support molecules can be added to the composition prior to the association of the cargo, DNA dendrimer, and optionally, the adaptor molecule. In some embodiments, support molecules can be added to the composition after the association of the cargo, DNA dendrimer, and optionally, the adaptor molecule.

In some embodiments, support molecules are comprised of biocompatible peptides, polymers, or both. In some embodiments, the support molecules can comprise a cell penetrating peptide (CPP) sequence, a nuclear localization signal (NLS) sequence, or both. In some embodiments, the CPP sequence is any CPP sequence disclosed herein. In some embodiments, the NLS sequence is any NLS sequence disclosed herein.

In some embodiments, the one or more support molecules have an amino acid sequence of ATPKKSTKKTPKKAKKATPKKSTKKTPKKAKK (SEQ ID NO: 12), WRRRGFGRRR (SEQ ID NO: 13), GRKKRRQRRRPQ (SEQ ID NO: 14), PKKKRKV (SEQ ID NO: 15), GLFHAIAIFIHGGWHGLIHGWYG (SEQ ID NO: 16), WEAALAEALAEALAEHLAEALAEALEALAA (SEQ ID NO: 17), HHHHHHHHHH (SEQ ID NO: 18), (KK)n, where n=2-15, or any combination thereof. In some embodiments, the support molecule has an amino acid sequence of GLFHAIAHFIHGGWHGLIHGWYGWSQPPKKKRKVATPKKSTKKTPKKAKKATPKKSTK KTPKKAKK (SEQ ID NO: 19). In some embodiments, the support molecule has an amino acid sequence of HHHHHHHHHHATPKKSTKKTPKKAKKATPKKSTKKTPKKAKK (SEQ ID NO: 20). In some embodiments, the support molecule has an amino acid sequence of GLFHAIAHFIHGGWHGLIHGWYGWSQPPKKKRKVGRKKRRQRRRPQWRRRGFGRRR (SEQ ID NO: 21). In some embodiments, the support molecule has an amino acid sequence of KKKKKKKKKKKKKKKKKKK (SEQ ID NO: 24). In some embodiments, the support molecule has an amino acid sequence of KKKKKKKKKKKKKKKKKKKKKKKKKKKKKKKKKKKKKKK (SEQ ID NO: 25). In some embodiments, the support molecule has an amino acid sequence of

(SEQ ID NO: 26)
KKKKKKKKKKKKKKKKKKKKKKKKKKK
KKKKKKKKKKKKKKKKKKKKKKKKKKK
KKKKKKKKKKKKKKKKKKKKKKKKKKK
KKKKKKKKKKKKKKKKKKKKKKKKKKK
KKKKKK.

In some embodiments, polyethylene glycol (PEG) can be added to any support molecule disclosed herein. The PEG molecule can be any known PEG in the art, including a variety of molecular weights. For example, in some embodiments, the PEG is PEG400 or PEG2000. In some embodiments, the combination of the PEG molecule and the support molecule increases circulation time of the support molecule, shields positive charges, enhances stability of the composition, or any combination thereof. In some embodiments, the PEG molecule is attached to the N-terminus of the support molecule.

In some embodiments, support molecules are added at a specific concentration to the composition. In some embodiments, a support molecule is added to the composition to bring the ionic balance of the overall composition within an acceptable Nitrogen/Phosphate (N/P) ratio. A N/P ration is the number of nitrogen groups in the support molecule relative to the number of phosphorus groups in any nucleic acid molecule in a composition. In some embodiments, the higher the N/P ratio, the more support molecule is added to a composition. In some embodiments, the N/P ratio is calculated using the following formula:

$$\frac{N}{P} = \frac{\frac{Weight of peptide / polymer ( ug )}{\begin{matrix}( Molecular weight of peptide / polymer ) / \\ ( number of positive charge )\end{matrix}}}{\frac{Weight of DNA ( ug )}{Mean of Molecular Weight of dNMPs}}$$

In some embodiments, the support molecule is added to the composition so that the ionic balance of the composition has a N/P ratio between 0.25 and 10. In some embodiments, the support molecule is added to the composition so that the ionic balance of the composition has a N/P ratio between 0.5 and 10. In some embodiments, the support molecule is added to the composition so that the ionic balance of the composition has a N/P ratio between 1 and 10. In some embodiments, the support molecule is added to the composition so that the ionic balance of the composition has a N/P ratio between 2 and 10. In some embodiments, the support molecule is added to the composition so that the ionic balance of the composition has a N/P ratio between 3 and 10. In some embodiments, the support molecule is added to the composition so that the ionic balance of the composition has a N/P ratio between 4 and 10. In some embodiments, the support molecule is added to the composition so that the ionic balance of the composition has a N/P ratio between 5 and 10. In some embodiments, the support molecule is added to the composition so that the ionic balance of the composition has a N/P ratio between 6 and 10. In some embodiments, the support molecule is added to the composition so that the ionic balance of the composition has a N/P ratio between 7 and 10. In some embodiments, the support molecule is added to the composition so that the ionic balance of the composition has a N/P ratio between 8 and 10. In some embodiments, the support molecule is added to the composition so that the ionic balance of the composition has a N/P ratio between 9 and 10. In some embodiments, the support molecule is added to the composition so that the ionic balance of the composition has a N/P ratio between 0.25 and 9. In some embodiments, the support molecule is added to the composition so that the ionic balance of the composition has a N/P ratio between 0.25 and 8. In some embodiments, the support molecule is added to the composition so that the ionic balance of the composition has a N/P ratio between 0.25 and 7. In some embodiments, the support molecule is added to the composition so that the ionic balance of the composition has a N/P ratio between 0.25 and 6. In some embodiments, the support molecule is added to the composition so that the ionic balance of the composition has a N/P ratio between 0.25 and 5. In some embodiments, the support molecule is added to the composition so that the ionic balance of the composition has a N/P ratio between 0.25 and 4. In some embodiments, the support molecule is added to the composition so that the ionic balance of the composition has a N/P ratio between 0.25 and 3. In some embodiments, the support molecule is added to the composition so that the ionic balance of the composition has a N/P ratio between 0.25 and 2. In some embodiments, the support molecule is added to the composition so that the ionic balance of the composition has a N/P ratio between 0.25 and 1. In some embodiments, the support molecule is added to the composition so that the ionic balance of the composition has a N/P ratio between 0.25 and 5.

### F. Modifications

Modified nucleic acids may be used throughout the compositions described herein, including the DNA dendrimers, cargoes, adaptor molecules, and support molecules. Non-limiting examples of such chemical modifications independently include without limitation phosphate backbone modification (e.g. phosphorothioate internucleotide linkages), nucleotide sugar modification (e.g., 2′-O-methyl nucleotides, 2′-O-allyl nucleotides, 2′-deoxy-2′-fluoro nucleotides, 2′-deoxyribonucleotides), nucleotide base modification (e.g., “universal base” containing nucleotides, 5-C-methyl nucleotides), and non-nucleotide modification (e.g., abasic nucleotides, inverted deoxyabasic residue) or a combination of these modifications. In addition, oligonucleotides having morpholino backbone structures (U.S. Pat. No. 5,034,506) or polyamide backbone structures (Nielsen et al., 1991, Science 254: 1497) may also be used. These and other chemical modifications can preserve biological activity of a nucleic acid in vivo while at the same time, dramatically increasing the serum stability, potency, duration of effect and/or specificity of these compounds. Nucleic acids containing modified internucleoside linkages may also be synthesized using reagents and methods that are well known in the art. For example, methods for synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH2-S—CH2), dimethylene-sulfoxide (—CH2-SO—CH2), dimethylene-sulfone (—CH2-SO2-CH2), 2′-O-alkyl, and 2′-deoxy-2′-fluoro phosphorothioate internucleoside linkages are well known in the art (see Uhlmann et al., 1990, Chem. Rev. 90:543-584; Schneider et al., 1990, Tetrahedron Lett. 31:335 and references cited therein).

The examples of oligonucleotide modifications described herein are not exhaustive and it is understood that the compositions includes additional modifications which serve to enhance the therapeutic or other properties of the oligonucleotides without appreciable alteration of the basic sequence of the oligonucleotide. Similarly, protein cargoes may be modified as described elsewhere herein.

Additionally, linkages described herein may be noncovalent or covalent. Covalent linkages include linkages susceptible to cleavage once internalized in a cell. Such linkages include pH-labile, photo-labile and radio-labile bonds and are well known in the art. Cargos may be linked to an oligonucleotide comprising a sequence that is substantially complementary to a binding arm to a sequence present in one of the binding arms on the surface of the DNA dendrimer or to a portion of single stranded sequence of any DNA-based carrier. The oligonucleotide may further comprise a nucleic acid cargo. The binding arm of a DNA dendrimer, or a portion of any DNA-based carrier, may also be designed to comprise a sequence complementary to a sequence in a known nucleic acid molecule (e.g., genomic DNA, cDNA, RNAs, plasmids, etc.) in order to link a nucleic acid cargo directly to the DNA dendrimer (via hydrogen bonding). A binding arm, branch or the body of a DNA dendrimer, or a portion of any DNA-based carrier, may also be designed to comprise a sequence that is a cargo (e.g., a DNA oligonucleotide).

### Pharmaceutical Compositions and Kits

In some embodiments, pharmaceutical compositions of the compositions are provided. In some embodiments, the composition further comprises a pharmaceutically acceptable carrier. For therapeutic use, the compositions may be prepared as pharmaceutical compositions containing an effective amount of the cargo, composition, domain, or molecule as an active ingredient in a pharmaceutically acceptable carrier. A “pharmaceutically acceptable carrier” refers to a diluent, adjuvant, excipient, or vehicle with which the active compound is administered. The pharmaceutically acceptable carrier is separate from a DNA dendrimer or other components as provided for herein. In some embodiments, the pharmaceutical composition does not have any additional pharmaceutically acceptable carriers. Such vehicles can be liquids, such as water and oils, including those of petroleum, animal, vegetable, or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating, and coloring agents, etc. The concentration of the molecules disclosed herein in such pharmaceutical formulation can vary widely, i.e., from less than about 0.5%, usually at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g., Remington: The Science and Practice of Pharmacy, 21st Edition, Troy, D. B. ed., Lipincott Williams and Wilkins, Philadelphia, PA 2006, Part 5, Pharmaceutical Manufacturing pp 691-1092, See especially pp. 958-989.

In some embodiments, any composition disclosed herein may also comprise suitable formulation agents known in the art, such as stabilizers, buffers, excipients, and the like that are different from a support molecule or other components as provided for herein. For example, suitable formulation agents include, but are not limited to purified water, calcium carbonate, calcium phosphate, various sugars, starches, cellulose derivatives, gelatin, polymers such as polyethylene glycols, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH7.0), 0.9% saline, 1.2% saline, silicone, waxes, petroleum jelly, polyethylene glycol, propylene glycol, liposomes, sugars such as mannitol and lactose, and other materials depending on the specific type of formulation used.

The mode of administration for therapeutic use of the compositions disclosed herein may be any suitable route that delivers the agent to the host, such as parenteral administration, e.g., intradermal, intramuscular, intraperitoneal, intravenous or subcutaneous, pulmonary; transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

Pharmaceutical compositions can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Such a kit can further comprise written information on indications and usage of the pharmaceutical composition.

### Methods

In some embodiments, methods of delivering a molecule of interest to a target cell is provided. In some embodiments, the molecule of interest is delivered to the nucleus of the target cell. In some embodiments, the methods comprise contacting the target cell with any of the compositions or pharmaceutical compositions disclosed herein. Without being bound to any theory, the targeting moiety can bind to the target cell to facilitate the composition to enter into the target cell, and wherein the cargo polynucleotide, can be transported to the nucleus of the target cell. In some embodiments, the cargo polynucleotide is linked to an adaptor molecule, such as any adaptor molecule disclosed herein. In some embodiments, the cargo polynucleotide, the adaptor molecule, or both further comprise a DTS, a NLS, or both, to assist with transport into the nucleus of the target cell. In some embodiments, the adaptor molecule comprises a CPP. In some embodiments, the cargo polynucleotide expresses the molecule of interest inside the nucleus of the target cell. In some embodiments, the composition of pharmaceutical composition can express more than one molecule of interest. In some embodiments, the promoter or promoters of the cargo polynucleotide are tissue specific promoters. In some embodiments, the targeting moiety is a tissue specific targeting moiety. In some embodiments, the composition or pharmaceutical composition comprises two or more tissue specific targeting moieties. In some embodiments, the targeting moieties can be of any type disclosed herein.

In some embodiments, methods of treating a disease are provided. In some embodiments, the methods comprise contacting the target cell with any of the compositions or pharmaceutical compositions disclosed herein. In some embodiments, the targeting moiety binds to the target cell to facilitate the composition to enter into the target cell. In some embodiments, he cargo polynucleotide can enter into the nucleus of the target cell. In some embodiments, the cargo polynucleotide is linked to the DNA dendrimer. In some embodiments, the cargo polynucleotide is linked to an adaptor molecule, such as any adaptor molecule disclosed herein. In some embodiments, the cargo polynucleotide, the adaptor molecule, or both further comprise a DTS, a NLS, or both, to assist with transport into the nucleus of the target cell. In some embodiments, the adaptor molecule comprises a CPP. In some embodiments, the cargo polynucleotide expresses the molecule of interest inside the nucleus of the target cell. In some embodiments, the composition of pharmaceutical composition can express more than one molecule of interest. In some embodiments, the promoter or promoters of the cargo polynucleotide are tissue specific promoters. In some embodiments, the targeting moiety is a tissue specific targeting moiety. In some embodiments, the composition or pharmaceutical composition comprises two or more tissue specific targeting moieties. In some embodiments, the targeting moieties can be of any type disclosed herein.

In some embodiments, the composition is administered to a subject according to a route of administration as provided for herein. In some embodiments, the composition is administered parenterally, such as intravenously.

In some embodiments, the cargo of the composition is a therapeutic agent, and the method is used to alleviate a disorder or disease or provide a prophylactic treatment for a disorder or disease. The method is carried out by administering any composition or pharmaceutical provided herein to an individual in need thereof. Without being bound to any theory, by avoiding or limiting lysosomal delivery and degradation of a cargo and successfully delivering the cargo to the nucleus of the cell, the compositions and pharmaceutical compositions provided for herein may enable the reduction in dose of a therapeutic, compared to prior art delivery methods. Reducing the dose also advantageously reduces the risk of potential side effects. In some embodiments, the therapeutic agent is a polypeptide or a small molecule drug. In other embodiments, the therapeutic agent comprises nucleic acid. The therapeutic molecule may be any therapeutic molecule that can be encoded in a polynucleotide. Non-limiting examples of types of therapeutic molecules that can be encoded in an expression cassette in the instant invention include, but are not limited to, polypeptide enzymes, cytokines, hormones, antibodies, such as intrabodies or scFvs, a suicide gene, such as HSV-TK, a molecule that inhibits vascularization, a molecule that increases vascularization, tumor suppressors, such as p53 and p21, pro-apoptotic molecules, such as TRAIL, transcription factors, receptors, ligands, immunogenic molecules, anti-proliferative molecules, agonists, antagonists, anti-inflammatory molecules, antibiotics, antidepressants, prodrugs, anti-hypertensives, anti-oxidants, and the like. The therapeutic molecule comprising a nucleic acid may be a nucleic acid that modulates the expression in vivo of a gene. Such nucleic acids include antisense molecules, siRNA and ribozymes.

Thus, the methods herein provides a novel therapeutic approach to a broad spectrum of diseases and conditions, including cancer or cancerous disease, infectious disease, ocular disease, cardiovascular disease, neurological disease, prion disease, inflammatory disease, autoimmune disease, metabolic disease, genetic disease, pulmonary disease, renal disease, liver disease, mitochondrial disease, endocrine disease, reproduction related diseases and conditions, graft vs host disease, and any other indications that can respond to the level of an expressed gene product in a cell or individual. In some embodiments, the disease is a genetic disease or disorder, including, but not limited to, spinocerebelar ataxia, dentatorubral-pallidoluysian atrophy, Huntington disease, muscular atrophy, machado joseph disease, choreoacanthocytosis, spastic paraplegia, dystrophia moytonica, fragile X syndrome, fragile X ataxia syndrome, spinocerebellar ataxia, frontotemporal dementia, amyotrophic lateral sclerosis, myotonic dystrophy, cystic fibrosis, and down syndrome. In some embodiments, the disease has been shown to be treatable or potentially treatable by gene therapy, including, but not limited to, multiple myeloma, B-cell lymphoma, melanoma, Leber congenital amaurosis, spinal muscular atrophy, lipoprotein lipase deficiency, metachromatic leukodystrophy, mantel cell lymphoma, large B-cell lymphoma, beta thalassemia, vascular endothelial growth factor peripheral artery disease, cerebral adrenoleukodystrophy, head and neck squamous cell carcinoma, adenosine deaminase deficiency, and B cell lymphoblastic leukemia.

The art is replete with exemplary molecules and associated diseases or disorders where a patient may benefit from the expression or inhibition of expression of one or more molecules. For instance, an assessment of expression changes in gene families in a variety of human cancers has been pursued (U.S. Pat. Appl. Pub. No. 20060168670). In addition, tissue-specific expression levels have been mapped for thousands of genes through expression profiling (Alon et al., 1999, Proc. Natl. Acad. Sci. USA 96:6745-50; Iyer et al., 1999, Science 283: 83-87; Khan et al., 1998, Cancer Res. 58: 5009-13; Lee et al., 1999, Science 285:1390-93; Wang et al., 1999, Gene 229:101-08; and Whitney et al., 1999, Ann. Neurol. 46:42). Thus, the skilled artisan is able to select molecules useful in the practice of the present invention without undue experimentation.

In some embodiments, a method of manufacturing a cargo polynucleotide is provided, the method comprising adding at least one promoter and at least one coding sequence encoding for at least one molecule of interest to a plasmid backbone to form a plasmid, then optionally contacting the plasmid with one or more restriction enzymes. In some embodiments, the plasmid is contacted with one or more restriction enzymes to form various topologies, including a nicked circular nucleotide, a linear nucleotide with a closed 5′ and 3′ end, a linear nucleotide with open 5′ and 3′ ends, or a linear nucleotide with one open and one closed end topology. In some embodiments, the cargo polynucleotide is then linked to an adaptor molecule, which is in turn linked to a DNA dendrimer to form the composition. In some embodiments, the plasmid is not contacted with one or more restriction enzymes to form a full circular nucleotide topology. In some embodiments, the cargo polynucleotide is then contacted with a DNA dendrimer linked to a targeting moiety in the presence of a support molecule capable, such that the cargo polynucleotide and the DNA dendrimer associated together to form the composition. In some embodiments, the support molecule is any support molecule described herein. In some embodiments, the support molecule condenses the size of the cargo polynucleotide, the DNA dendrimer, or both.

In some embodiments, the plasmid containing the at least one coding sequence is amplified by traditional bacterial methods, for example, E. coli. In some embodiments, the plasmid containing the at least one coding sequence is amplified by rolling circle amplification, or other non-bacterial methods. For example, amplification can occur using enzymatic synthesis of covalently closed linear DNA, as described in U.S. Pat. No. 11,149,302 and U.S. Patent Publication No. US2019/0185924, both of which are incorporated by reference in their entirety.

### EMBODIMENTS

Embodiments provided herein also include, but are not limited to, the following:

1. A composition comprising a DNA dendrimer linked to, or associated with:
a targeting moiety; and
an adaptor molecule-cargo polynucleotide complex.
   2. The composition of embodiment 1, wherein the DNA dendrimer is linked to the adaptor molecule-cargo polynucleotide complex through an electrostatic interaction, a covalent bond, a non-covalent bond, or a hydrogen bond with the adaptor molecule.
   3. The composition of embodiments 1 or 2, wherein the cargo polynucleotide comprises at least one promoter and at least one coding sequence encoding for at least one molecule of interest, wherein the cargo polynucleotide is a nicked circular polynucleotide, a linear polynucleotide with a closed 5′ and 3′ end, a linear polynucleotide with open 5′ and 3′ ends, or a linear polynucleotide with one open and one closed end, wherein the one open and one closed end can be at either the 5′ and 3′ of the polynucleotide.
   4. The composition of embodiment 2, wherein the cargo polynucleotide further comprises at least one DNA targeting sequence (DTS).
   5. The composition of embodiment 4, wherein the at least one DTS is located upstream of the at least one promoter.
   6. The composition of embodiment 4, wherein the at least one DTS is located downstream of the at least one promoter.
   7. The composition of any one of embodiments 4-6, wherein the cargo polynucleotide comprises two or more DTSs.
   8. The composition of embodiment 7, wherein the two or more DTSs are located next to, or approximately next to, each other.
   9. The composition of embodiment 7, wherein the two or more DTSs are not located next to, or approximately next to, each other.
   10. The composition of any one of embodiments 1-5, wherein the cargo polynucleotide further comprises at least one nuclear localization signal sequence (NLS).
   11. The composition of embodiment 10, wherein the at least one NLS is located upstream of the at least one promoter.
   12. The composition of embodiment 10, wherein the at least one NLS is located downstream of the at least one promoter.
   13. The composition of any one of embodiments 6-8, wherein the cargo polynucleotide comprises at least one DTS and at least one NLS.
   14. The composition of any one of embodiments 1-9, wherein the adaptor molecule comprises a DNA dendrimer binding sequence (DBS) and a cargo binding region (CBR).
   15. The composition of embodiment 14, wherein the adaptor is linked to the DNA dendrimer through the DBS.
   16. The composition of embodiments 14 or 15, wherein the DBS comprises a nucleic acid sequence that is complimentary to a nucleic acid sequence on the DNA dendrimer.
   17. The composition of any one of embodiments 14-16, wherein the DBS comprises a nucleic acid sequence of TAGAGGTAACAACTAGCGTACAA (SEQ ID NO: 3), CCTCAGCTTGTACTCTAGTTGTTACCTCTAATGCTGGACCTCAGC (SEQ ID NO: 22), or CCTCAGCACCCTACAGAGTAACCTAGATTGATCAAACACCTCAGC (SEQ ID NO: 23).
   18. The composition of any one of embodiments 14-17, wherein the DBS further comprises a polythymine sequence at either the 5′ or 3′ end of the DBS.
   19. The composition of embodiment 18, wherein the DBS comprises a nucleic acid sequence of TAGAGGTAACAACTAGCGTACAATTTTTTTTTT (SEQ ID NO: 4).
   20. The composition of any one of embodiments 14-19, wherein the cargo binding region (CBR) can be linked to the cargo polynucleotide by DNA ligation, i.e., is linked to the polynucleotide through a covalent bond, such as phosphodiester bond.
   21. The composition of any one of embodiments 14-19, wherein the CBR can be linked to the cargo polynucleotide by chemical coupling, i.e. is linked to the polynucleotide through a covalent bond, such as a disulfide bond.
   22. The composition of any one of embodiments 14-21, wherein the cargo binding region comprises at least one cysteine.
   23. The composition of any one of embodiments 14-22, wherein the adaptor molecule further comprises a tag, such as, for example, a tag that can be used as an affinity tag to isolate/purify the composition complex.
   24. The composition of embodiment 23, wherein the tag is located between the DBS and the CBR.
   25. The composition of embodiment 24, wherein the tag is a polyhistidine tag.
   26. The composition of any one of embodiments 14-25, wherein the adaptor molecule further comprises at least one nuclear localization signal sequence (NLS).
   27. The composition of embodiment 26, wherein the at least one NLS is located between or overlaps with the DBS and the cargo binding region.
   28. The composition of embodiments 26 or 27, wherein the at least one NLS comprises one or more NLS sequences disclosed herein.
   29. The composition of any one of embodiments 26-28, wherein the at least one NLS comprises an amino acid sequence of YPDEVKRKKKP (SEQ ID NO: 1).
   30. The composition of any one of embodiments 26-28, wherein the at least one NLS comprises an amino acid sequence of

(SEQ ID NO: 2)
SLLESPFDKPDEVKRKKKPPTSHQSDATAEDDSSSKKK

31. The composition of any one of embodiments 26-30, wherein the at least one NLS further comprises at least one spacer located either before or after the at least one NLS.
32. The composition of embodiment 31, wherein the at least one NLS comprises two spacers located before or after the at least one NLS, such as one before the NLS and one after the NLS, or both spacers before or after the NLS.
33. The composition of embodiment 31 or 32, wherein the spacer or spacers comprise polyethylene glycol (PEG), propylene glycol alginate (PGA), PEG-polylactic acid (PLA), poly lactic-co-glycolic acid (PGLA), or any combination thereof.
34. The composition of embodiment 31 or 32, wherein spacer or spacers comprise a polyglycine sequence, optionally with alanine and/or serine residues.
35. The composition of embodiment 34, wherein the spacer or spacers have the amino acid sequence of GGGG (SEQ ID NO: 5).
36. The composition of any one of embodiments 14-35, wherein the adaptor molecule further comprises at least one DNA targeting sequence (DTS).
37. The composition of embodiment 36, wherein the at least one DTS is located between or overlaps with the DBS and the CBR.
38. The composition of any one of embodiments 14-37, wherein the adaptor molecule further comprises a cell penetrating peptide sequence (CPP).
39. The composition of embodiment 38, wherein the CPP is located between or overlapping with the DBS and the CBR.
40. The composition of embodiments 38 or 39, wherein the CPP comprises a CPP disclosed herein.
41. The composition of any one of embodiments 38-40, wherein the CPP comprises an amino acid sequence of QPRRRPRRKKRG (SEQ ID NO: 6).
42. The composition of any one of embodiments 14-41, wherein the adaptor molecule further comprises at least one cleavage site.
43. The composition of embodiment 42, wherein the at least one cleavage site is located between, or overlaps with, the DBS and the CBR.
44. The composition of embodiments 42 or 43, wherein the cleavage site is located adjacent to the DBS.
45. The composition of any one of embodiments 42-44, wherein the cleavage site is a val-cit linker.
46. The composition of any one of embodiments 14-45, wherein the adaptor molecule further comprises at least one flexible linker.
47. The composition of embodiment 46, wherein the at least one flexible linker is located between the DNS and the CBR.
48. The composition of embodiments 46 or 47, wherein the at least one flexible linker is selected from the group consisting of polyethylene glycol (PEG), propylene glycol alginate (PGA), PEG-polylactic acid (PLA), poly lactic-co-glycolic acid (PGLA), (GG)n, (GGGGS)n, or (GGGGA)n, wherein each n is independently, 1-5.
49. The composition of any one of embodiments 1-47, wherein the composition further comprises a support molecule.
50. The composition of embodiment 49, wherein the support molecule is linked to the DNA dendrimer.
51. The composition of embodiment 49, wherein the support molecule is not linked to the DNA dendrimer.
52. The composition of any one of embodiments 49-51, wherein the support molecule condenses the size of the cargo polynucleotide, the DNA dendrimer, or both.
53. The composition of any one of embodiments 49-52, wherein the support molecule can assist in nuclear delivery.
54. The composition of any one of embodiments 49-53, wherein the support molecule can protect the cargo polynucleotide, the DNA dendrimer, or both from nuclease degradation.
55. The composition of any one of embodiments 49-54, wherein the support molecule can enhance the transfection efficiency of the composition.
56. The composition of any one of embodiments 49-55, wherein the support molecule comprises an amino acid sequence of ATPKKSTKKTPKKAKKATPKKSTKKTPKKAKK (SEQ ID NO: 12), WRRRGFGRRR (SEQ ID NO: 13), GRKKRRQRRRPQ (SEQ ID NO: 14), PKKKRKV (SEQ ID NO: 15), GLFHAIAHFIHGGWHGLIHGWYG (SEQ ID NO: 16), WEAALAEALAEALAEHLAEALAEALEALAA (SEQ ID NO: 17), HHHHHHHHHH (SEQ ID NO: 18), (KK)q, wherein q is 2-15.
57. The composition of any one of embodiments 49-56, wherein the support molecule comprises an amino acid sequence of

(SEQ ID NO: 19)
GLFHAIAHFIHGGWHGLIHGWYGWSQPPKKKRK
VATPKKSTKKTPKKAKKATPKKSTKKTPKKAKK.

58. The composition of any one of embodiments 49-56, wherein the support molecule comprises an amino acid sequence of

(SEQ ID NO: 20)
HHHHHHHHHHATPKKSTKKTPKKAKKATPKKSTKKTPKKAKK.

59. The composition of any one of embodiments 49-56, wherein the support molecule comprises an amino acid sequence of

(SEQ ID NO: 21)
GLFHAIAHFIHGGWHGLIHGWYGWSQPP
KKKRKVGRKKRRQRRRPQWRRRGFGRRR.

60. The composition of any one of embodiments 49-56, wherein the support molecule comprises an amino acid sequence of KKKKKKKKKKKKKKKKKKK (SEQ ID NO: 24).
61. The composition of any one of embodiments 49-56, wherein the support molecule comprises an amino acid sequence of

(SEQ ID NO: 25)
KKKKKKKKKKKKKKKKKKKKKKKKKKKKKKKKKKKKKKK.

62. The composition of any one of embodiments 49-56, wherein the support molecule comprises an amino acid sequence of

(SEQ ID NO: 26)
KKKKKKKKKKKKKKKKKKKKKKKKKKK
KKKKKKKKKKKKKKKKKKKKKKKKKKK
KKKKKKKKKKKKKKKKKKKKKKKKKKK
KKKKKKKKKKKKKKKKKKKKKKKKKKK
KKKKKK.

63. The composition of any one of embodiments 1-62, wherein the targeting moiety is selected from the group consisting of an antibody, a naturally-occurring ligand for the receptor or a functional derivative thereof, a vitamin, a hormone, a small molecule mimetic of a naturally-occurring ligand, a peptide, a polypeptide, a peptidomimetic, a carbohydrate, a lipid, an aptamer, a nucleic acid, a toxin, a component of a microorganism, any other molecule provided it binds specifically to the cell surface molecule and induces endocytosis of the bound moiety, or any combination thereof.
64. A composition comprising a cargo polynucleotide, a support molecule, and a DNA dendrimer linked to a targeting moiety.
65. The composition of embodiment 64, wherein the targeting moiety is selected from the group consisting of an antibody, a naturally-occurring ligand for the receptor or a functional derivative thereof, a vitamin, a hormone, a small molecule mimetic of a naturally-occurring ligand, a peptide, a polypeptide, a peptidomimetic, a carbohydrate, a lipid, an aptamer, a nucleic acid, a toxin, a component of a microorganism, any other molecule provided it binds specifically to the cell surface molecule and induces endocytosis of the bound moiety, or any combination thereof.
66. The composition of embodiment 64 or 65, wherein:
the cargo polynucleotide comprises at least one promoter and at least one coding sequence encoding for at least one molecule of interest, and, optionally, a DNA binding sequence (DBS),
wherein the cargo polynucleotide is full circular polynucleotide (e.g., plasmid), nicked circular polynucleotide, a linear polynucleotide with closed 5′ and 3′ end, a linear polynucleotide with open 5′ and 3′ ends, or a linear polynucleotide with one open and one closed end,
wherein the one open and one closed end can be at either the 5′ and 3′ of the cargo polynucleotide.
   67. The composition of embodiment 66, wherein the cargo polynucleotide a closed circle polynucleotide.
   68. The composition of embodiment 66, wherein the cargo polynucleotide is a nicked circular polynucleotide.
   69. The composition of any one of embodiments 64-68, wherein the cargo polynucleotide is not covalently linked to the DNA dendrimer.
   70. The composition of any one of embodiments 64-69, wherein the cargo polynucleotide further comprises at least one DNA targeting sequence (DTS).
   71. The composition of embodiment 70, wherein the DTS is located within the cargo polynucleotide.
   72. The composition of embodiment 70, wherein the DTS is linked to the cargo polynucleotide.
   73. The composition of embodiment 72, wherein the cargo polynucleotide is a nicked circular polynucleotide, and the DTS is linked to the cargo polynucleotide at the single stranded DNA break (i.e., the location of the nick).
   74. The composition of any one of embodiments 64-73, wherein the cargo polynucleotide further comprises at least one nuclear localization signal sequence (NLS).
   75. The composition of any one of embodiments 64-74, wherein the cargo polynucleotide comprises at least one DTS and at least one NLS.
   76. The composition of any one of embodiments 66-75, wherein the DBS comprises a nucleic acid sequence of TAGAGGTAACAACTAGCGTACAA (SEQ ID NO: 3), CCTCAGCTTGTACTCTAGTTGTTACCTCTAATGCTGGACCTCAGC (SEQ ID NO: 22), or CCTCAGCACCCTACAGAGTAACCTAGATTGATCAAACACCTCAGC (SEQ ID NO: 23).
   77. The composition of embodiment 76, wherein the DBS further comprises a polythymine sequence at either the 5′ or 3′ end of the DBS.
   78. The composition of embodiment 77, wherein the DBS comprises a nucleic acid sequence of TAGAGGTAACAACTAGCGTACAATTTTTTTTTT (SEQ ID NO: 4).
   79. The composition of any one of embodiments 64-78, wherein the support molecule is linked to the DNA dendrimer.
   80. The composition of any one of embodiments 64-79, wherein the support molecule is not linked to the DNA dendrimer.
   81. The composition of any one of embodiments 64-80, wherein the support molecule condenses the size of the cargo polynucleotide, the DNA dendrimer, or both.
   82. The composition of any one of embodiments 64-81, wherein the support molecule can assist in nuclear delivery.
   83. The composition of any one of embodiments 64-82, wherein the support molecule can protect the cargo polynucleotide, the DNA dendrimer, or both from nuclease degradation.
   84. The composition of any one of embodiments 64-83, wherein the support molecule can enhance the transfection efficiency of the composition.
   85. The composition of any one of embodiments 64-84, wherein the presence of the support molecule facilitates the association of the cargo polynucleotide and the DNA dendrimer to each other.
   86. The composition of any one of embodiments 64-85, wherein the support molecule has a positive charge.
   87. The composition of any one of embodiments 64-86, wherein the support molecule comprises an amino acid sequence of ATPKKSTKKTPKKAKKATPKKSTKKTPKKAKK (SEQ ID NO: 12), WRRRGFGRRR (SEQ ID NO: 13), GRKKRRQRRRPQ (SEQ ID NO: 14), PKKKRKV (SEQ ID NO: 15), GLFHAIAHFIHGGWHGLIHGWYG (SEQ ID NO: 16), WEAALAEALAEALAEHLAEALAEALEALAA (SEQ ID NO: 17), HHHHHHHHHH (SEQ ID NO: 18), (KK)q, or any combination thereof, wherein q is 2-15.
   88. The composition of any one of embodiments 64-87, wherein the support molecule comprises an amino acid sequence of

(SEQ ID NO: 19)
GLFHAIAHFIHGGWHGLIHGWYGWSQPPK
KKRKVATPKKSTKKTPKKAKKATPKKSTK
KTPKKAKK.

89. The composition of any one of embodiments 64-87, wherein the support molecule comprises an amino acid sequence of

(SEQ ID NO: 20)
HHHHHHHHHHATPKKSTKKTPKKAKKATPKKSTKKTPKKAKK.

90. The composition of any one of embodiments 64-87, wherein the support molecule comprises an amino acid sequence of

(SEQ ID NO: 21)
GLFHAIAHFIHGGWHGLIHGWYGWSQPP
KKKRKVGRKKRRQRRRPQWRRRGFGRRR.

91. The composition of any one of embodiments 64-87, wherein the support molecule comprises an amino acid sequence of KKKKKKKKKKKKKKKKKKK (SEQ ID NO: 24).
92. The composition of any one of embodiments 64-87, wherein the support molecule comprises an amino acid sequence of

(SEQ ID NO: 25)
KKKKKKKKKKKKKKKKKKKKKKKKKKKKKKKKKKKKKKK

93. The composition of any one of embodiments 64-87, wherein the support molecule comprises an amino acid sequence of

(SEQ ID NO: 26)
KKKKKKKKKKKKKKKKKKKKKKKKKKK
KKKKKKKKKKKKKKKKKKKKKKKKKKK
KKKKKKKKKKKKKKKKKKKKKKKKKKK
KKKKKKKKKKKKKKKKKKKKKKKKKKK
KKKKKK.

94. The composition of any one of embodiments 64-93, wherein the support molecule further comprises a polyethylene glycol (PEG) molecule linked to the support molecule.
95. The composition of embodiment 94, wherein the polyethylene molecule is PEG2000.
96. The composition of embodiment 94 or 95 wherein the PEG molecule is attached to the N-terminus of the support molecule.
97. The composition of any one of embodiments 1-97, wherein the coding sequence of the cargo polynucleotide encodes for an antibody, an enzyme, a protein, a miRNA, a siRNA, an antisense RNA, and the like.
98. The compositions of any one of embodiments 1-97, wherein the nitrogen/phosphate (N/P) ratio of the composition is between 0.5 and 10.
99. The composition of embodiment 98, wherein the N/P ratio of the composition is between 2 and 5.
100. A pharmaceutical composition comprising a composition of any one of embodiments 1-99 and a pharmaceutically acceptable carrier.
101. A method of delivering a molecule of interest to the nucleus of a target cell, the method comprising contacting the target cell with the composition of any one of embodiments 1-99 or the pharmaceutical composition of embodiment 100, wherein the targeting moiety binds to the target cell.
102. The method of embodiment 101, wherein the cargo polynucleotide expresses the molecule of interest in the nucleus of the target cell.
103. The method of embodiments 101 or 102, wherein the cargo polynucleotide expresses two or more molecules of interest in the nucleus of the target cell.
104. The method of any one of embodiments 101-103, wherein the cargo polynucleotide comprises a tissue specific promoter.
105. The method of any one of embodiments 101-104, wherein the targeting moiety is a tissue specific targeting moiety.
106. The method of any one of embodiments 101-105, wherein the composition or pharmaceutical composition comprises two or more targeting moieties.
107. A method of treating a disease, the method comprising administering the composition of any one of embodiments 1-99 or the pharmaceutical composition of embodiment 100 to a subject to treat the disease, wherein the targeting moiety binds to the target cell.
108. The method of embodiment 107, wherein the cargo polynucleotide expresses the molecule of interest in the nucleus of the target cell.
109. The method of embodiments 107 or 108, wherein the cargo polynucleotide expresses two or more molecules of interest in the nucleus of the target cell.
110. The method of any one of embodiments 107-109, wherein the cargo polynucleotide comprises a tissue specific promoter.
111. The method of any one of embodiments 107-110, wherein the targeting moiety is a tissue specific targeting moiety.
112. The method of any one of embodiments 107-111, wherein the composition or pharmaceutical composition comprises two or more targeting moieties.
113. A plasmid comprising a plasmid backbone comprising at least two enzyme restriction recognition sites, at least one promoter, at least one coding sequence encoding for at least one molecule of interest, and, optionally, a DNA dendrimer binding sequence (DBS), wherein the plasmid is capable of forming a cargo polynucleotide with various structures depending on whether the plasmid has one or more, or none of, a 5′ end and a 3′ end.
114. The plasmid of embodiment 113, wherein the plasmid is a full circular polynucleotide, a nicked circular nucleotide, a linear nucleotide with a closed 5′ and 3′ end, a linear nucleotide with open 5′ and 3′ ends, and a linear nucleotide with one open and one closed end.
115. The plasmid of embodiment 113, wherein the nicked circular nucleotide, linear nucleotide with a closed 5′ and 3′ end, linear nucleotide with open 5′ and 3′ ends, and linear nucleotide with one open and one closed end are formed by cutting the plasmid with one or more restriction enzymes.
116. The plasmid of any one of embodiments 113-115, wherein the plasmid further comprises at least one DNA targeting sequence (DTS).
117. The plasmid of embodiment 116, wherein the at least one DTS is located in the plasmid backbone, i.e., between, or overlapping with, the at least two enzyme restriction recognition sites.
118. The plasmid of embodiments 116 or 117, wherein the at least one DTS is located upstream of the at least one promoter.
119. The plasmid of embodiments 116 or 117, wherein the at least one DTS is located downstream of the at least one promoter.
120. The plasmid of any one of embodiments 113-119, wherein the cargo polynucleotide further comprises at least one nuclear localization signal sequence (NLS).
121. The plasmid of embodiment 120, wherein the at least one NLS is located upstream of the at least one promoter.
122. The plasmid of embodiment 120, wherein the at least one NLS is located downstream of the at least one promoter.
123. The plasmid of any one of embodiments 116-122, wherein the cargo polynucleotide comprises at least one DTS and at least one NLS.
124. The composition of any one of embodiments 116-123, wherein the DBS comprises a nucleic acid sequence of TAGAGGTAACAACTAGCGTACAA (SEQ ID NO: 3), CCTCAGCTTGTACTCTAGTTGTTACCTCTAATGCTGGACCTCAGC (SEQ ID NO: 22), or CCTCAGCACCCTACAGAGTAACCTAGATTGATCAAACACCTCAGC (SEQ ID NO: 23).
125. The composition of embodiment 124, wherein the DBS further comprises a polythymine sequence at either the 5′ or 3′ end of the DBS.
126. The composition of embodiment 125, wherein the DBS comprises a nucleic acid sequence of TAGAGGTAACAACTAGCGTACAATTTTTTTTTT (SEQ ID NO: 4).
127. The plasmid of any one of embodiments 116-126, wherein the at least one promoter is a tissue specific promoter.
128. The plasmid of any one of embodiments 116-126, wherein the plasmid comprises polynucleotide sequences encoding for two or more molecules of interest.
129. A method of manufacturing the composition of any one of embodiments 1-63, the method comprising:
contacting the plasmid of any one of embodiments 113-129 with one or more restriction enzymes that cut at the restriction enzyme recognition sites to form a nicked circular polynucleotide, a linear polynucleotide with a closed 5′ and 3′ end, a linear polynucleotide with open 5′ and 3′ ends, or a linear polynucleotide with one open and one closed end,
linking the cargo polynucleotide to the adaptor molecule to form an adaptor molecule-cargo polynucleotide complex, and
linking the adaptor molecule-cargo polynucleotide complex and a targeting moiety to a DNA dendrimer to form the composition.
   130. A method of manufacturing the composition of any one of embodiments 64-99, the method comprising:
contacting the plasmid of any one of embodiments 113-129 that comprise a DNA dendrimer binding sequence (DBS) with one or more restriction enzymes that cut at the restriction enzyme recognition sites to form a nicked circular polynucleotide, a linear polynucleotide with a closed 5′ and 3′ end, a linear polynucleotide with open 5′ and 3′ ends, or a linear polynucleotide with one open and one closed end, and
linking the cargo polynucleotide and a targeting moiety to a DNA dendrimer to form the composition.
   131. A method of manufacturing the composition of any one of embodiments 64-99, the method comprising
contacting a circular uncut plasmid of any one of embodiments 113-129 with a DNA dendrimer linked to a targeting moiety with a support molecule capable of condensing the size of the cargo polynucleotide and the DNA dendrimer, such that the cargo polynucleotide and the DNA dendrimer are associated together to form the composition.
   132. A kit comprising a composition of embodiments 1-99, a pharmaceutical composition of embodiment 100, a plasmid of any one of embodiments 113-129, or some combination thereof.

### EXAMPLES

The following examples are provided for the purpose of illustration only and the claims should in no way be construed as being limited to these examples, but rather should be construed to encompass any and all variations which become evident as a result of the teaching provided herein. Those of skill in the art will readily recognize a variety of non-critical parameters that could be changed or modified to yield essentially similar results.

### Example 1: Characterization of Multiple Topologies for Polynucleotide Cargo

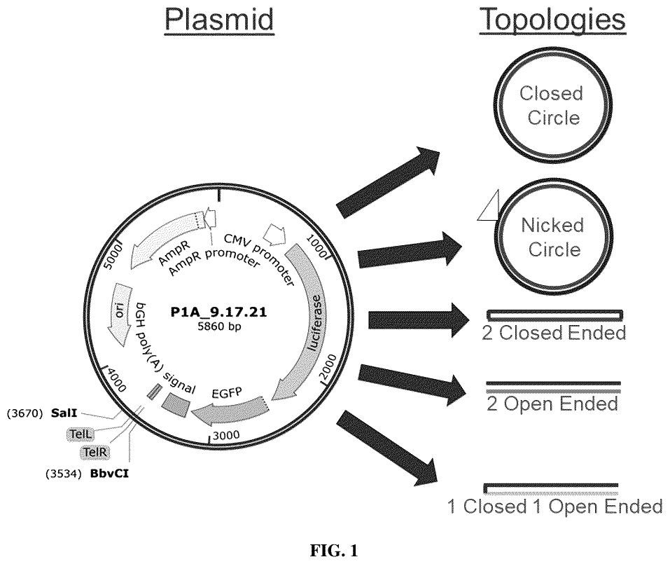

To determine how the topology structures of various polynucleotide cargo can affect expression efficiency, a gene cassette containing luciferase and eGFP driven by a CMV promoter was inserted into a plasmid backbone comprising multiple restriction enzyme recognition sites, including SalI, BbcCI, and Tel. To generate various topologies, the resulting plasmid was cut with the following restriction enzymes as listed in Table 1. A depiction of the various topologies is shown in FIG. 1.

TABLE 1
Plasmid Topologies

| Topology Name | Restriction Enzymes Used |
|---|---|
| Full circular | none |
| Nicked circular | Nt.BbvCl |
| Linear, 2 closed ends | TelN Protelomerase |
| Linear, 2 open ends | BbvCl |
| Linear, 1 closed end, 1 open end | TelN Protelomerase; BbvCl |

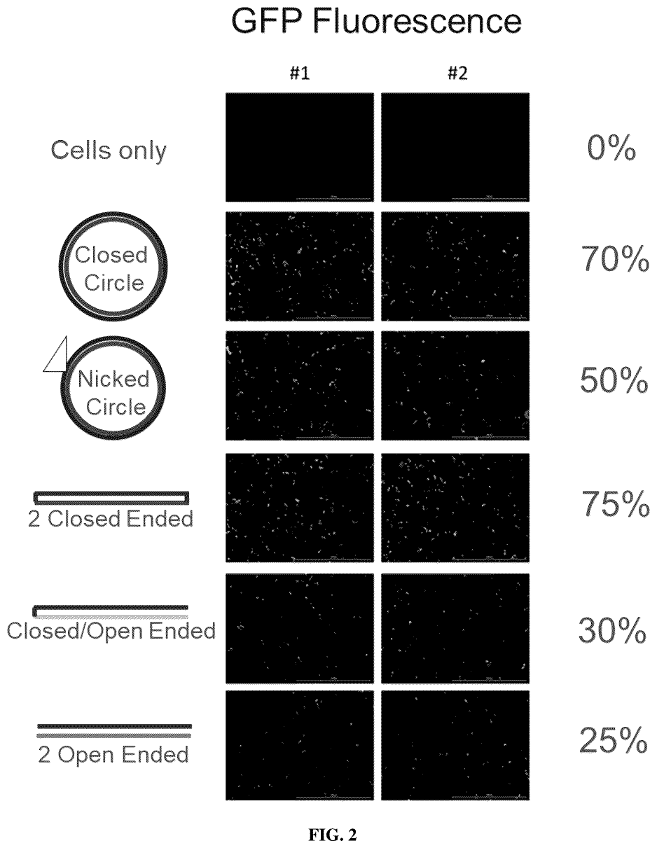

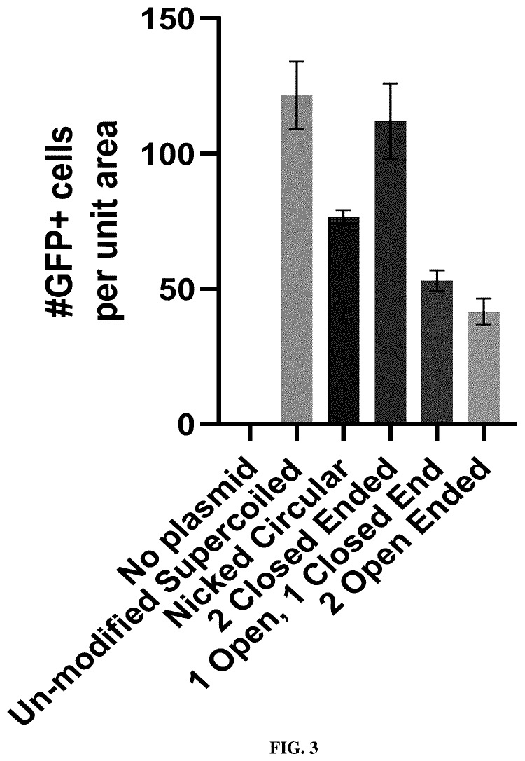

Once formed, the various polynucleotide cargoes were transfected into CHO-K1 cells using equal molar amount of each topological variation. GFP expression was measured oved time using cell imaging and quantitative fluorescence measurement. The percent of GFP positive cells and the overall fluorescent intensity were compared. Results shown in FIG. 2 and FIG. 3. The unmodified full circular (i.e., plasmid) and the linear with 2 closed ends topologies generally had the best overall fluorescence activity, however all the topologies had active fluorescence, which is useful in a change in overall activity of a specific therapeutic is needed.

### Example 2: Nuclear Localization for Non-Dividing Cells

In many cases, it is critical that a cargo polynucleotide be delivered to the nucleus of a target cell for expression of the encoded molecules of interest. When cells divide, the nuclear membrane natural breaks down, allowing direct access from the cytoplasm. However, in the absence of mitosis, the nuclear membrane remains intact and largely impermeable to plasmids. A potential strategy is to include at least one copy of a DNA targeting sequence (DTS), a nuclear localization sequence (NLS), or both in the cargo polynucleotide, an adaptor molecule that can be associated with the cargo polynucleotide, or both. In a healthy cytoplasm, transcription factors, including NLS, can bind to a DTS and recruit the cargo for nuclear transport. Likewise, a NLS can recruit the necessary importins directly, reading the cargo for nuclear transport. To test this general strategy, a DTS was included in a polynucleotide plasmid expressing eGFP and compared to a plasmid expressing eGFP without DTS in three different cell lines: fast growing CHO-K1, medium growth A427, and relatively slow dividing C2C12 differentiated myoblasts.

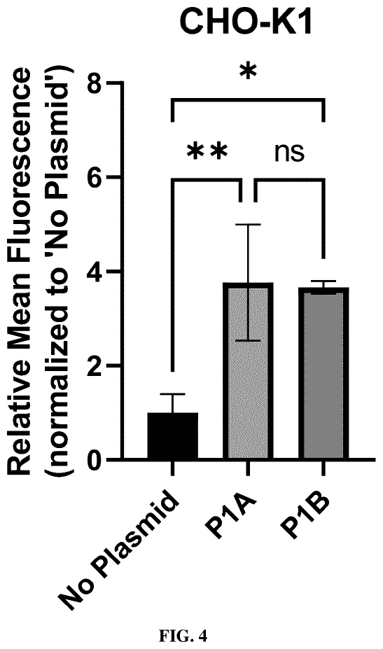

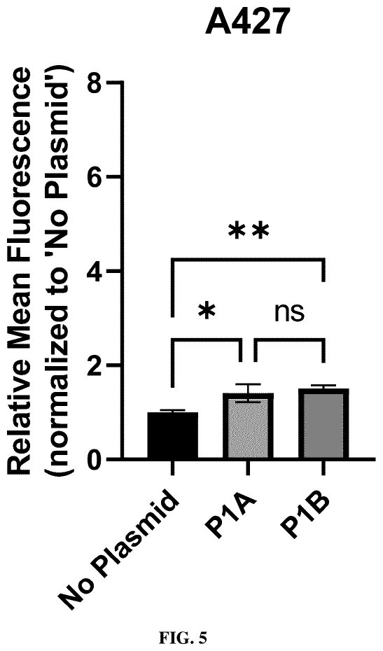

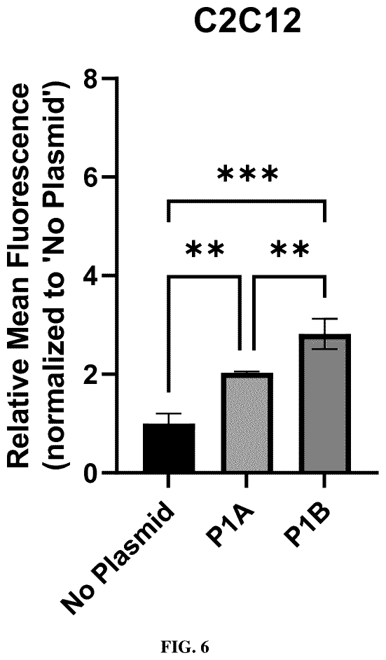

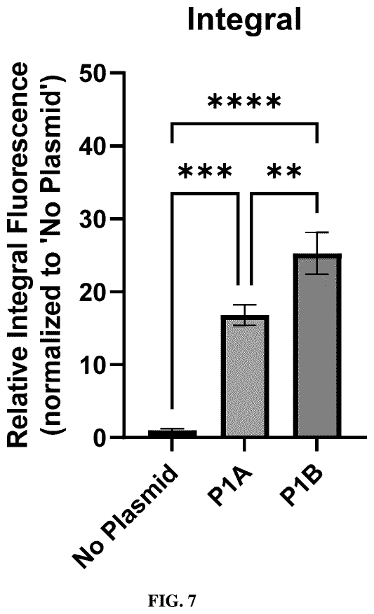

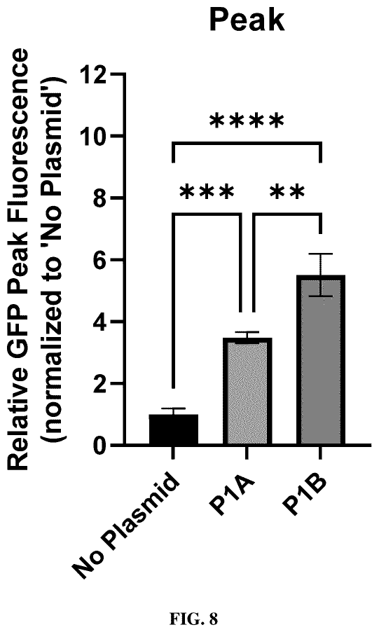

In general, both the no DTS plasmid (P1A) and the DTS plasmid (P1B) were able to express GFP in the CHO-K1 (FIG. 4) and the A427 cell lines (FIG. 5). This was expected, as both cell lines undergo enough mitosis to allow ready access to the cell nuclei. However, in the C2C12 cell line, where the no-to-slow growth rates limit access to the otherwise intact nuclei, the DTS containing plasmid cargo had significantly better expression than the control plasmid in relative mean fluorescence (FIG. 6), relative integral fluorescence (FIG. 7), and relative peak fluorescence (FIG. 8). For FIGS. 4-8, roughly 5,000 cells per well were plated, and imaging and quantification of fluorescence was measured 24 hours after initial transfection. Significance was determined using one-way ANOVA, and Tukey's multiple comparison's test between groups; *p<0.05, **p<0.01, ***p<0.001, and ****p<0.0001.

### Example 3: Additional Characterization of Multiple Topologies for Polynucleotide Cargo

Additional polynucleotide cargo plasmids were made and tested.

Reporter Constructs

All reporter constructs contain a bacterial selection sequence (antibiotic resistance, origin of replication (ORI)), SV40-DTS, codon-optimized firefly luciferase (Luc2_CO), codon-optimized enhanced green fluorescent protein (eGFP_CO), 2A self-cleaving peptide (P2A), and the bovine growth hormone polyadenylateion (bgh-PolyA) signal. In addition, a TelN protelomerase sequence is used in conjunction with one of two DBS sequences (SEQ ID NO: 22 or SEQ ID NO: 23) that allows for plasmid attachment to the DNA dendrimer nanoparticle via complementary hybridization. Two nickase enzyme sites flanking the DBS enzyme are used to expose the single-stranded DNA (ssDNA) sequence to the chosen attachment arm of the synthetic scaffold.

Three reporter constructs were made. Reporter constructs 1 and 2 share a cytomegalovirus (CMV) enhancer and promoter which is known for robust and ubiquitous expression in mammalian cell lines. Both reporter constructs also share a Kozak sequence for initiation of eukaryotic translation. Reporter construct 1 has a DBS sequence of SEQ ID NO: 22 while reporter construct 2 has the DBS sequence of SEQ ID NO: 23. Reporter construct 3 has a DBS sequence of SEQ ID NO: 22, but a different promoter: the muscle specific promoter mCK8e. All three reporter constructs have various cut sites for restriction enzymes and double nickase sites were placed for plasmid manipulation. The area containing the selected enzyme cut sites and attachment sequences is designated as the “CB Backbone Elements” region. An additional feature in this region is the TelN protelomerase recognition site. This sequence is a palindrome and upon recognition by the enzyme forms two covalently closed ends (denoted “TelR” and “TelL”) at the site of cleavage.

Additionally, a molecule of interest construct was made, where the DNA insert encoding the construct is more than 10K base pairs. This construct has many of the same sequences found in the reporter constructs: the bacterial selection portion, SV40-DTS, mCK8e promoter, Kozak sequence, and TelN protelomerase site. An additional promoter, the T7 promoter, is incorporated before the coding sequence for enhanced bacterial expression. Similar restriction enzymes and double nickase sites are present near DBS sequence (SEQ ID NO: 22).

Methods

Restriction enzymes used only with reporter constructs are denoted with “†” and those used only with the 10K insert construct are denoted with “‡”. A nickase denoted as “Nt” is only used with constructs with the SEQ ID NO: 22 DBS site. A nickase denoted as “Nb” is only used with constructs with the SEQ ID NO: 23 DBS site. Any enzymes or nickases without symbols are universal and can be used for any construct listed.

1. Open Nicked Circle (OpC)

Constructs with the SEQ ID NO: 22 DBS site are incubated with Nt.BbvCI in a ratio of 1 μg of plasmid to 1 unit (U) of enzyme (10,000 U/mL, New England Biolabs, R0632L, Lot #10141032). Those with the SEQ ID NO: 23 DBS site are incubated with Nb.BbvCI in a ratio of 1 μg of plasmid to 1 U of enzyme (10,000 U/mL, New England Biolabs, R0631L, Lot #10150965). The reaction is then incubated for 1 hour at 50° C. at 300 revolutions per minute (rpm).

The appropriate capture sequence for the DBS site is then added in a 20 pmol excess to the plasmid. The reactions are then heated to 90° C. for 2 minutes to allow for the nicked strand to dissociate from the plasmid. After, reactions are cooled slowly to room temperature over the course of 3.5 hours for the capture sequence to bind to the nicked sequence and leave the attachment site open.

Ion-exchange chromatography (IEX) is then utilized to separate the nicked plasmid from the captured nicked sequence. Fractions containing the peaks of interest are then combined and the OpC plasmid is extracted using ethanol (EtOH) precipitation. Pellets are then dried and reconstituted in the buffer of choice.

2. Linear Nicked (LN)

Constructs are incubated with TelN Protelomerase in a ratio of 1 μg of plasmid to 1 U of enzyme (20,000 U/mL, New England Biolabs, M0651B-HC2, Lot #10151691). Reactions are incubated for 4 hours at 30° C. and 300 rpm. A buffer swap into nuclease-free water is carried out to remove the Triton-X from the initial buffer.

TelN linearized constructs with the SEQ ID NO: 22 DBS site are incubated with Nt.BbvCI in a ratio of 1 μg of plasmid to 1 unit (U) of enzyme (10,000 U/mL, New England Biolabs, R0632L, Lot #10141032). Those with the SEQ ID NO: 23 DBS site are incubated with Nb.BbvCI in a ratio of 1 μg of plasmid to 1 U of enzyme (10,000 U/mL, New England Biolabs, R0631L, Lot #10150965). The reaction is then incubated for 1 hour at 50° C. at 300 rpm.

The appropriate capture sequence for the DBS site is then added in a 20 μmol excess to the plasmid. The reactions are then heated to 90° C. for 2 minutes to allow for the nicked strand to dissociate from the plasmid. After, reactions are cooled slowly to room temperature over the course of 3.5 hours for the capture sequence to bind to the nicked sequence and leave the attachment site open.

IEX is then utilized to separate the nicked plasmid from the captured nicked sequence. Fractions containing the peaks of interest are then combined and the LN plasmid is extracted using EtOH precipitation. Pellets are then dried and reconstituted in the buffer of choice.

3. 1-Open, 1-Closed (1co)

Constructs are incubated with TelN Protelomerase in a ratio of 1 μg of plasmid to 1 U of enzyme (20,000 U/mL, New England Biolabs, M0651B-HC2, Lot #10151691). Reactions are incubated for 4 hours at 30° C. and 300 rpm. A buffer swap into nuclease free water is carried out to remove the Triton X from the initial buffer.

MfeI† (20,000 U/mL, New England Biolabs, R3589L, Lot #10150327) or EagI‡ (100,000 U/mL, New England Biolabs, R3505M, Lot #10157687) are incubated with the TelN linearized constructs in a ratio of 1 μg plasmid to 1 U of enzyme. Reactions are incubated for 1 hour at 37° C. at 300 rpm. An EtOH precipitation is used to precipitate the 1-open ended, linearized plasmid before the nickase reaction.

Constructs with the SEQ ID NO: 22 DBS site are incubated with Nt.BbvCI in a ratio of 1 μg of plasmid to 1 unit (U) of enzyme (10,000 U/mL, New England Biolabs, R0632L, Lot #10141032). Those with the SEQ ID NO: 23 DBS site are incubated with Nb.BbvCI in a ratio of 1 μg of plasmid to 1 U of enzyme (10,000 U/mL, New England Biolabs, R0631L, Lot #10150965). The reaction is then incubated for 1 hour at 50° C. at 300 rpm.

The appropriate capture sequence for the DBS site is then added in a 20 μmol excess to the plasmid. The reactions are then heated to 90° C. for 2 minutes to allow for the nicked strand to dissociate from the plasmid. After, reactions are cooled slowly to room temperature over the course of 3.5 hours for the capture sequence to bind to the nicked sequence and leave the attachment site open.

IEX is then utilized to separate the nicked plasmid from the captured nicked sequence. Fractions containing the peaks of interest are then combined and the 1co plasmid is extracted using ethanol EtOH precipitation. Pellets are then dried and reconstituted in the buffer of choice.

4. 2-Open Ends (2oe)

MfeI† (20,000 U/mL, New England Biolabs, R3589L, Lot #10150327) or EagI‡ (100,000 U/mL, New England Biolabs, R3505M, Lot #10157687) are incubated with the constructs in a ratio of 1 μg plasmid to 1 U of enzyme. Reactions are incubated for 1 hour at 37° C. at 300 rpm. An EtOH precipitation is used to precipitate the 2oe linearized plasmid before reconstitution in its chosen buffer.

Constructs with the SEQ ID NO: 22 DBS site are incubated with Nt.BbvCI in a ratio of 1 μg of plasmid to 1 unit (U) of enzyme (10,000 U/mL, New England Biolabs, R0632L, Lot #10141032). Those with the SEQ ID NO: 23 DBS site are incubated with Nb.BbvCI in a ratio of 1 μg of plasmid to 1 U of enzyme (10,000 U/mL, New England Biolabs, R0631L, Lot #10150965). The reaction is then incubated for 1 hour at 50° C. at 300 revolutions per minute rpm.

The appropriate capture sequence for the DBS site is then added in a 20 μmol excess to the plasmid. The reactions are then heated to 90° C. for 2 minutes to allow for the nicked strand to dissociate from the plasmid. After, reactions are cooled slowly to room temperature over the course of 3.5 hours for the capture sequence to bind to the nicked sequence and leave the attachment site open.

IEX is then utilized to separate the nicked plasmid from the captured nicked sequence. Fractions containing the peaks of interest are then combined and the 1co plasmid is extracted using ethanol EtOH precipitation. Pellets are then dried and reconstituted in the buffer of choice.

Confirmation Results

Reporter Construct 1 was used for all experiments unless otherwise noted.

1. Confirmation of OpC Plasmid.

Using a restriction fragment length polymorphism (RFLP) analysis the efficacy of the double nickase was assessed on a 15% TBE-Urea denaturing polyacrylamide gel. The plasmid pre-modification showed no gel bands, while the post-nickase plasmid showed a 38 bp band, which correlates with expected band size from the cut. For the RFLP, TelN was used to open the plasmid before an additional restriction enzyme cut further up from the nicked site. This resulted in 2 smaller fragments (47 and 58 bp) from the top nicked strand, and one longer fragment (209 bp) from the bottom strand. Additionally, the 38 bp band from the nickase is present since this analysis was run before the IEX purification. These bands correlated with the expected sizes from the cuts.

To observe if the modified plasmids were still functional HepG2 cells were transfected with Reporter Construct 1 in the OpC topology with Lipofectamine2000 (Catalog #11668019, ThermoFisher Scientific, Waltham, MA). Cells were seeded at ˜2.5×104 cells/well one day prior to treatment. Cells were imaged after 48 hours for fluorescence (excitation/emission 488/509 nm) due to eGFP expression. The fluorescence signal was quantitated using the sum of integrated fluorescent intensity, which allows for the individual pixels to be represented without bias. The results showed that the Reporter Construct 1 has a high distribution of fluorescence signal.

2. Confirmation of Linear Nicked (LN) Plasmid.

For the RFLP of the LN plasmid, only an additional restriction enzyme upstream from the nickase site was necessary since the plasmid was already treated with TelN. The 38 bp band is present from the nickase as well as the 47, 58, and 209 bp bands, as expected.

3. Confirmation of the 1-Open, 1-Closed (1co) Plasmid

The RFLP performed on the 1co Plasmid showed that prior to the addition of the nickase, the 132 bp band for the removed closed end and 30 bp for the check cut were present as expected. Post-addition of the nickase RFLP with 132, 48, 46, and 38 bp bands produced from the removal of the closed end, smaller fragments of the nicked strand, and the excised piece of DNA, all as expected.

### Example 4: Characterization of Support Molecules for Complexation and Protection of Polynucleotide Cargo

Nucleases are enzymes that cleave phosphodiester bonds between the nucleotides of nucleic acids. They are often found in DNA repair mechanisms such as replication proofreading, Okazaki fragment processing, mismatch repairs, base-excision repair, nucleotide-excision repair, and double-strand break repair. For nucleic acid-based gene delivery methods nucleases can be a large inhibitor of activity by destroying the payload before it can be transcribed (in the case of DNA) or translated (for ribonucleic acids, RNA).

An additional factor to take into consideration for in vivo delivery is the size of the nanocarrier with versus without its payload. Pore sizes vary from cell type to cell type and will only permit materials of the same size to be taken up. The size of a DNA dendrimer nanocarrier is approximately 60 nm. Depending on the topology of the payload this can increase the particle size dramatically. Previously measured linearized pDNA was approximately 750 nm in length post hybridization to the DNA dendrimer. To enable delivery to cells with smaller pore sizes, the particle must be reduced or compacted.

In addition to size, particle size and protection can increase durability of the scaffold and cargo. Support molecules can both compact nanoparticles and shield them from degrading enzymes. These support molecules can be either peptide-based, polymer-based, or a hybrid of both in order to obtain the desired properties.

Several peptide-based and polymer-based support molecules were tested, as listed in Table 1 below. Peptide-based support molecules carry a mix of positively charged and neutral residues which allows for nucleic acid complexation, but also provides protection against nucleases. Polymer-based support molecules are comprised of various sized poly-L-pysines (PPLs) and have high complexation success rate but can have decreased ability to protect nucleic acid cargo from degradation by nucleases. Additionally, a PEG2000 was added to the N-terminus of Exc 1 (SEQ ID NO: 19) to form a hybrid support molecule (not shown in Table 1).

TABLE 1

| Name | SEQ ID NO | Sequence |
|---|---|---|
| Exc 1 | 19 | GLFHAIAHFIHGGWHGLIH |
| | | GWYGWSQPPKKKRKVATPK |
| | | KSTKKTPKKAKKATPKKST |
| | | KKTPKKAKK |
| Exc 2 | 20 | HHHHHHHHHHATPKKSTKK |
| | | TPKKAKKATPKKSTKKTPK |
| | | KAKK |
| Exc 3 | 21 | GLFHAIAHFIHGGWHGLIH |
| | | GWYGWSQPPKKKRKVGRKK |
| | | RRQRRRPQWRRRGFGRRR |
| PLL P1 | 24 | KKKKKKKKKKKKKKKKKKK |
| PLL P2 | 25 | KKKKKKKKKKKKKKKKKKK |
| | | KKKKKKKKKKKKKKKKKKK |
| | | K |
| PLL P3 | 26 | KKKKKKKKKKKKKKKKKKK |
| | | KKKKKKKKKKKKKKKKKKK |
| | | KKKKKKKKKKKKKKKKKKK |
| | | KKKKKKKKKKKKKKKKKKK |
| | | KKKKKKKKKKKKKKKKKKK |
| | | KKKKKKKKKKKKKKKKKKK |

The amount of support molecule used to complex and protect the plasmid constructs is determined by the N/P ratio, the formula for which is disclosed herein. The higher the N/P ratio, the more support molecule is present in the final composition. Once the N/P ratio is determined, the support molecule and plasmid solutions can be combined. The solutions are mixed with 10× phosphate buffered saline (PBS) providing salt to assist in complexation and nuclease free water (NFW) to make up the total volume. Support molecules can be used with DNA concentrations from about 0.05 μg/μL to about 0.5 μg/μL. For example, to achieve an N/P ratio of 3 with 300 ug of a plasmid solution with a concentration of 1.0 μg/μL and 1300 μg of a support molecule solution of 5.0 μg/μL with a final DNA concentration of 0.5 μg/μL, 300 μL of plasmid solution, 260 μL of support molecule solution, 6 μL of 10×PBS, and 34 μL of NFW are combined to a total volume of 600 μL. This solution is then mixed at 300 rpm for 30 minutes at room temperature.

To determine if the support molecules were successful at complexing the plasmid nucleic acids, final products were run on 1.1% agarose gels to observe if free plasmid was still present after the reaction. Results showed that Exc 1 showed partial complexation at a N/P ratio of 1 or 2, but complete complexation at a N/P ratio of 5 and 8. Exc 2 showed no complexation at a N/P ration of 1 and a partial complexation at a N/P ratio of 5, while Exc 3 showed no complexation at a N/P ratio of 1 or 5. Polymer-based support molecules perform at lower N/P ratios due to their uniformly positive sequences. PLL P1, PLL P2, and PLL P3 all showed complete complexation at N/P ratios as low as 0.5. The hybrid PEG-Exc 1 support molecule shown complexation at similar N/P ratios to Exc 1 by itself.

Nuclease protection was determined using a DNase I assay. Samples were subjected to 5 U of DNase I (M030S, New England Biolabs) for 30 minutes at 37° C. This amount was determined by titrating the amount of DNase I was required to fully degrade a 0.5 ug of plasmid, the maximum amount of DNA able to be handled by the cleanup kits. Samples were then purified with a commercially available kit from New England Biolabs (T1030L). Following cleanup, samples were run on 1.1% agarose gels to observe if complexes, plasmid, or both were remaining. Experiments were run with Exc 1 complexed with plasmid DNA at N/P rations 5 and 8. As a control, plasmid complexed with a transfection agent TurboFect™ (R0531, ThermoFisher Scientific), was also included as it has demonstrated ability to complex as well. While all three solutions complexed with the DNA plasmid, TurboFect™ was unable to protect the plasmid from degradation. Exc 1, at both N/P ratios shows little to no degradation. Additional experiments with the PPL support molecules show that they have weaker DNase protection. The PEG-Exc 1 hybrid protects equivalently with Exc 1, indicating that PEG does not impact the stability of the support molecule.

To determine if the PEG-Exc 1 hybrid support molecule would cause any cytotoxicity, a CellTiter-Glo® Luminescent Cell Viability Assay (G7570, Promega) was performed on treated murine cells (C2C12, CRL-1772, ATCC). Cells were incubated with support molecule encapsulate conjugations (nanocarrier, plasmid, targeting moieties, and support molecule) with the dose based on the plasmid mass (0.2 μg and 1.0 μg per well). Samples were tested in quintuplet. At 0.2 μg per well, there was no statistical loss in viability from either PEG-Exc 1 or TurboFect™ as compared the control cells. At 1.0 μg per well both PEG-Exc 1 and TurboFect™ impacted viability compared with control cells.

Additional support molecules can be designed. Modifications to the number of H1 sequences may compact the nucleic acids and the N/P ratio, and overall stabilizer content, may be reduced through their addition. Different CPPs, such as arginine-8, may be added in alter transfection both in vitro and in vivo models. Likewise, alternative NLS sequences can be investigated to enhance nuclear delivery. Additionally, peptide-targeting sequences can be added to the base stabilizer during synthesis or can be chemically added through functional groups (e.g., Click chemistry, amides, cross-linking). Finally, specific areas of the support molecules could be made neutral or positive for zonal segregation of charge for complexation and new functional groups could be added at the termini or distributed throughout the chain. For example, these could include binding sites for small molecules, antibodies, and peptide sequences.

1. A composition comprising a DNA dendrimer linked to, or associated with:
a targeting moiety; and
an adaptor molecule-cargo polynucleotide complex.

2-132. (canceled)
